(19) United States
(12) Patent Application Publication
(10) Pub. No.: US 2023/0089351 A1
(43) Pub. Date: Mar. 23, 2023

(54) METHODS RELATED TO BIOACTIVE AGENTS THAT CONVERT FROM ANIONS TO MOLECULES

(71) Applicant: NATURAL EXTRACTION SYSTEMS, LLC, Boulder, CO (US)

(72) Inventor: Douglas G. Metcalf, Boulder, CO (US)

(73) Assignee: NATURAL EXTRACTION SYSTEMS, LLC, Boulder, CO (US)

(21) Appl. No.: 17/797,088

(22) PCT Filed: Feb. 2, 2021
(86) PCT No.: PCT/US2021/016263

§ 371 Date: Aug. 2, 2022

Related U.S. Application Data
(60) Provisional application No. 62/969,616, filed on Feb. 3, 2020.
(60) Provisional application No. 62/971,802, filed on Feb. 7, 2020.
(60) Provisional application No. 63/003,752, filed on Apr. 1, 2020.
(60) Provisional application No. 63/059,823, filed on Jul. 31, 2020.

(51) Int. Cl. A61K 31/05; A61K 31/353; A61K 31/567; A61K 31/57

(52) U.S. Cl. CPC A61K 31/05; A61K 31/353; A61K 31/567; A61K 31/57

(57) ABSTRACT

Various aspects of this patent document relate to bioactive compositions comprising anions that are converted into hydrophobic bioactive molecules either ex vivo prior to administration to a subject or in situ subsequent to administration to a subject. Such compositions display improved bioavailability and pharmacokinetics relative to compositions that comprise the hydrophobic bioactive molecules instead of the anions.

### CROSS-REFERENCE TO RELATED APPLICATIONS

This patent application claims priority to U.S. Provisional Patent Application No. 62/969,616, filed Feb. 3, 2020, U.S. Provisional Patent Application No. 62/971,802, filed Feb. 7, 2020, U.S. Provisional Patent Application No. 63/003,752, filed Apr. 1, 2020, and U.S. Provisional Patent Application No. 63/059,823, filed Jul. 31, 2020, each of which is incorporated by reference in its entirety.

### BACKGROUND

The “Rule of Five” states that druglike molecules generally have octanol-water partition coefficients of no greater than 5. A generally-applicable strategy to overcome this feature of the Rule of Five is desirable.

### SUMMARY

An octanol-water partition coefficient of greater than 5 limits solubility in bodily fluids, which limits bioavailability. Many classes of hydrophobic bioactive molecules can be converted into anions that display improved octanol-water partition coefficients using simple acid/base chemistry.

Such anions convert back into their parent bioactive molecules upon administration to a subject. Various aspects of this patent document relate to the discovery that the anionic form of a bioactive agent can overcome the octanol-water partition coefficient limitation set forth in the Rule of Five.

Novel aqueous compositions that utilize the inventive chemistry in relation to specific classes of bioactive agents are disclosed, for example, in U.S. Pat. Nos. 10,555,914 and 10,609,944. This patent document discloses methods related to additional classes of bioactive agents.

### DETAILED DESCRIPTION

Various aspects of this patent document relate to a method to sedate or anesthetize a subject, comprising providing a composition comprising 2,6-diisopropylphenolate and administering the composition to the subject in an amount that is effective to sedate or anesthetize the subject.

“Comprise” and “comprising” refer to an open set such that a composition that comprises 2,6-diisopropylphenolate can also comprise water.

In some embodiments, the composition comprises water; the 2,6-diisopropylphenolate is dissolved in the water; and the water has a pH that is greater than 8.5 when the composition is provided. In some specific embodiments, the water has a pH that is greater than 9.5 when the composition is provided. In some very specific embodiments, the water has a pH that is greater than 10.5 and no greater than 13.5 when the composition is provided.

In some embodiments, the composition comprises the 2,6-diisopropylphenolate at a concentration of at least 15 grams per liter.

In some embodiments, the composition lacks triglycerides, fatty acids, and phospholipids at a combined concentration greater than 50 grams per liter.

In some embodiments, the composition comprises 2,6-diisopropylphenol, and the composition comprises the 2,6-diisopropylphenolate at a greater molar concentration than the 2,6-diisopropylphenol when the composition is provided.

In some embodiments, the method comprises contacting the 2,6-diisopropylphenolate with a Brønsted acid to convert the 2,6-diisopropylphenolate into 2,6-diisopropylphenol. In some specific embodiments, the method comprises contacting the composition with a liquid comprising bicarbonate to convert the 2,6-diisopropylphenolate into 2,6-diisopropylphenol.

In some embodiments, the method comprises converting the 2,6-diisopropylphenolate into 2,6-diisopropylphenol in situ subsequent to administering the composition to the subject. In some embodiments, the amount comprises at least 0.5 micrograms and no greater than 5 milligrams of 2,6-diisopropylphenolate per kilogram bodyweight of the subject per minute.

In some embodiments, the method comprises converting the 2,6-diisopropylphenolate into 2,6-diisopropylphenol ex vivo prior to administering the composition to the subject. In some embodiments, the amount comprises at least 0.5 micrograms and no greater than 5 milligrams of 2,6-diisopropylphenol per kilogram bodyweight of the subject per minute.

Various aspects of this patent document relate to a method to modulate international normalized ratio (“INR”) in a subject, comprising providing a composition comprising 2-oxo-3-(1-phenylpropyl)-2H-chromen-4-oxide and administering the composition to the subject in an amount that is effective to either increase INR to greater than 1.2 in the subject or maintain INR at greater than 1.2 in the subject.

“INR” refers to prothrombin time ratio raised to the International Sensitivity Index for the prothrombin time assay, in which prothrombin time ratio is the prothrombin time of the subject divided by the prothrombin time of a normal control for the relevant assay.

In some embodiments, the amount is effective to increase INR to at least 1.5 and no greater than 4.0 in the subject or maintain INR at at least 1.5 and no greater than 4.0 in the subject.

In some embodiments, the amount is effective to either prophylactically prevent or treat (a) thromboembolic events, (b) venous thrombus, (c) pulmonary embolism, (d) thromboembolic complications associated with atrial fibrillation or cardiac valve replacement in the subject, (e) myocardial infarction, (f) ischemic stroke, (g) ischemia-related death, or (h) two or more of the foregoing in the subject.

“Treat” refers to at least one of: to cure a health condition; to increase the probability that a health condition will be cured; to shorten the time over which a health condition is cured; to increase the probability that the time necessary to cure a health condition will be shortened; to decrease the severity of a health condition; to increase the probability that the severity of a health condition will decrease; to shorten the time over which the severity of a health condition is decreased; to increase the probability that the time necessary to decrease the severity of a health condition will be shortened; to inhibit a health condition from worsening; to increase the probability that a health condition will not worsen; to delay the worsening of a health condition; to increase the probability that the worsening of a health condition will be delayed; to inhibit the occurrence or recurrence of a health condition; to decrease the probability that a health condition will occur or reoccur; to delay the onset of a health condition; to increase the probability that the onset of a health condition will be delayed; to alleviate at least one symptom of a health condition; to increase the probability that at least one symptom of a health condition will be alleviated; to shorten the time over which at least one symptom of a health condition is alleviated; to increase the probability that the time necessary to alleviate at least one symptom of a health condition will be shortened; to decrease the severity of at least one symptom of a health condition; to increase the probability that the severity of at least one symptom of a health condition will be decreased; to shorten the time over which the severity of at least one symptom of a health condition is decreased; to increase the probability that the time necessary to decrease the severity of at least one symptom of a health condition will be shortened; to inhibit at least one symptom of a health condition from worsening; to increase the probability that at least one symptom of a health condition will not worsen; to delay the worsening of at least one symptom of a health condition; to increase the probability that the worsening of at least one symptom of a health condition will be delayed; to inhibit at least one symptom of a health condition from occurring or reoccurring; to decrease the probability that at least one symptom of a health condition will occur or reoccur; to delay the onset of at least one symptom of a health condition; and to increase the probability that the onset of at least one symptom of a health condition will be delayed.

In some embodiments, the composition comprises 2-oxo-3-(1-phenylpropyl)-2H-chromen-4-ol, and the composition comprises the 2-oxo-3-(1-phenylpropyl)-2H-chromen-4-oxide at a greater molar concentration than the 2-oxo-3-(1-phenylpropyl)-2H-chromen-4-ol when the composition is provided.

In some embodiments, the method comprises contacting the 2-oxo-3-(1-phenylpropyl)-2H-chromen-4-oxide with a Brønsted acid to convert the 2-oxo-3-(1-phenylpropyl)-2H-chromen-4-oxide into 2-oxo-3-(1-phenylpropyl)-2H-chromen-4-ol.

In some embodiments, the method comprises converting the 2-oxo-3-(1-phenylpropyl)-2H-chromen-4-oxide into 2-oxo-3-(1-phenylpropyl)-2H-chromen-4-ol in situ subsequent to administering the composition to the subject. In some embodiments, the amount comprises at least 0.1 milligrams and no greater than 20 milligrams of 2-oxo-3-(1-phenylpropyl)-2H-chromen-4-oxide per day.

In some embodiments, the method comprises converting the 2-oxo-3-(1-phenylpropyl)-2H-chromen-4-oxide into 2-oxo-3-(1-phenylpropyl)-2H-chromen-4-ol ex vivo prior to administering the composition to the subject. In some embodiments, the amount comprises at least 0.1 milligrams and no greater than 20 milligrams of 2-oxo-3-(1-phenylpropyl)-2H-chromen-4-ol per day.

In some embodiments, the composition is formulated for oral administration; the composition is formulated to allow the conversion of the 2-oxo-3-(1-phenylpropyl)-2H-chromen-4-oxide into 2-oxo-3-(1-phenylpropyl)-2H-chromen-4-ol before the 2-oxo-3-(1-phenylpropyl)-2H-chromen-4-oxide reaches the stomach of the subject; and the composition is formulated to allow absorption of the 2-oxo-3-(1-phenylpropyl)-2H-chromen-4-ol by the epithelial lining of the gastrointestinal tract between the lips and the stomach, excluding the stomach and the outer surfaces of the lips, and including the esophagus and the inner surfaces of the lips.

In some embodiments, the administering is inhalational administering or injecting.

In some embodiments, the composition is a liquid, and the 2-oxo-3-(1-phenylpropyl)-2H-chromen-4-oxide is dissolved in the liquid.

Various aspects of this patent document relate to a method to increase estrogen in a subject, comprising providing a composition comprising an estrogen anion and administering the composition to the subject in an amount that is effective to increase estrogen in the subject, wherein the estrogen anion is (8R,9S,13S,14S,17S)-17-hydroxy-13-methyl-6,7,8,9,11,12,14,15,16,17-decahydrocyclopenta[a]phenanthrene-3-oxide; (8R,9 S,13 S,14S,17R)-17-ethynyl-17-hydroxy methyl-7,8,9,11,12,14,15,16-octahydro-6H-cyclopenta[a]phenanthrene-3-oxide; or (8R,9S,13 S,14S,16R,17R)-16,17-dihydroxy-13-methyl-6,7,8,9,11,12,14,15,16,17-decahydrocyclopenta[a]phenanthrene-3-oxide.

In some embodiments, the method comprises contacting the estrogen anion with a Brønsted acid to convert the estrogen anion into an estrogen molecule.

Various aspects of this patent document relate to a method to administer an estrogen to a subject, comprising providing a composition comprising an estrogen anion and orally administering the composition to the subject, wherein: the estrogen anion has a conjugate acid that is an estrogen molecule; the estrogen molecule has an acid dissociation constant in water of at least 50 femtomolar and no greater than 50 nanomolar for conversion of the estrogen molecule into the estrogen anion; the composition is formulated to allow the conversion of the estrogen anion into the estrogen molecule before the estrogen anion reaches the stomach of the subject; and the composition is formulated to allow absorption of the estrogen molecule by the epithelial lining of the gastrointestinal tract between the lips and the stomach, excluding the stomach and the outer surfaces of the lips, and including the esophagus and the inner surfaces of the lips.

In some embodiments, the estrogen anion is (8R,9S,13S,14S,17S)-13-methyl-17-[(1-oxopentyl)oxy]-6,7,8,9,11,12,14,15,16,17-decahydrocyclopenta[a]phenanthrene-3-oxide, and the estrogen molecule is (8R,9S,13S,14S,17S)-13-methyl-17-[(1-oxopentyl)oxy]-6,7,8,9,11,12,14,15,16,17-decahydrocyclopenta[a]phenanthren-3-ol; the estrogen anion is (8R,9S,13S,14S,17S)-13-methyl-17-[(1-oxo-3-cyclopentylpropyl)oxy]-6,7,8,9,11,12,14,15,16,17-decahydrocyclopenta[a]phenanthrene-3-oxide, and the estrogen molecule is (8R,9S,13S,14S,17S)-13-methyl-17-[(1-oxo-3-cyclopentylpropyl)oxy]-6,7,8,9,11,12,14,15,16,17-decahydrocyclopenta[a]phenanthren-3-ol; the estrogen anion is (8R,9S,13S,14S,17S)-13-methyl-17-[(1-oxoheptypoxy]-6,7,8,9,11,12,14,15,16,17-decahydrocyclopenta[a]phenanthrene-3-oxide, and the estrogen molecule is (8R,9S,13S,14S,17S)-13-methyl-17-[(1-oxoheptyl)oxy]-6,7,8,9,11,12,14,15,16,17-decahydrocyclopenta[a]phenanthren-3-ol; the estrogen anion is (8R,9S,13S,14S,17S)-13-methyl-17-[(1-oxoundecanyl)oxy]-6,7,8,9,11,12,14,15,16,17-decahydrocyclopenta[a]phenanthrene-3-oxide, and the estrogen molecule is (8R,9S,13S,14S,17S)-13-methyl-17-[(1-oxoundecanyl)oxy]-6,7,8,9,11,12,14,15,16,17-decahydrocyclopenta[a]phenanthren-3-ol; the estrogen anion is (8R,9S,13S,14S,17R)-17-hydroxy-13-methyl-6,7,8,9,11,12,14,15,16,17-decahydrocyclopenta[a]phenanthrene-3-oxide, and the estrogen molecule is (8R,9S,13S,14S,17R)-13-methyl-6,7,8,9,11,12,14,15,16,17-decahydrocyclopenta[a]phenanthrene-3,17-diol; the estrogen anion is (8S,9S,11S,13S,14S,17R) ethynyl-17-hydroxy-11-methoxy-13-methyl-7,8,9,11,12,14,15,16-octahydro-6H-cyclopenta[a]phenanthrene-3-oxide, and the estrogen molecule is (8S,9S,11S,13S,14S,17R) ethynyl-11-methoxy-13-methyl-7,8,9,11,12,14,15,16-octahydro-6H-cyclopenta[a]phenanthren-3,17-diol; the estrogen anion is (8R,9S,13S,14S,16S,17R)-16,17-dihydroxy-13-methyl-6,7,8,9,11,12,14,15,16,17-decahydrocyclopenta[a]phenanthrene-3-oxide, and the estrogen molecule is (8R,9S,13S,14S,16S,17R)-13-methyl-6,7,8,9,11,12,14,15,16,17-decahydrocyclopenta[a]phenanthrene-3,16,17-triol; the estrogen anion is (8R,9S,13S,14S,16S,17S)-16,17-dihydroxy-13-methyl-6,7,8,9,11,12,14,15,16,17-decahydrocyclopenta[a]phenanthrene-3-oxide, and the estrogen molecule is (8R,9S,13S,14S,16S,17S)-13-methyl-6,7,8,9,11,12,14,15,16,17-decahydrocyclopenta[a]phenanthrene-3,16,17-triol; the estrogen anion is (8R,9S,13S,14S)-17-oxo-13-methyl-7,8,9,11,12,14,15,16-octahydro-6H-cyclopenta[a]phenanthrene-3-oxide, and the estrogen molecule is (8R,9S,13S,14S)-17-oxo-13-methyl-7,8,9,11,12,14,15,16-octahydro-6H-cyclopenta[a]phenanthrene-3-ol; the estrogen anion is (9S,13S,14S)-17-oxo-13-methyl-9,11,12,14,15,16-hexahydro-6H-cyclopenta[a]phenanthrene-3-oxide, and the estrogen molecule is (9S,13 S,14S)-17-oxo-13-methyl-9,11,12,14,15,16-hexahydro-6H-cyclopenta[a]phenanthrene-3-ol; the estrogen anion is (13S,14S)-17-oxo-13-methyl-7,11,12,14,15,16-hexahydro-6H-cyclopenta[a]phenanthrene-3-oxide, and the estrogen molecule is (13S,14S)-17-oxo-13-methyl-7,11,12,14,15,16-hexahydro-6H-cyclopenta[a]phenanthrene-3-ol; the estrogen anion is (13S,14S)-17-oxo-13-methyl-12,14,15,16-tetrahydro-11H-cyclopenta[a]phenanthrene-3-oxide, and the estrogen molecule is (13S,14S)-17-oxo-13-methyl-12,14,15,16-tetrahydro-11H-cyclopenta[a]phenanthrene-3-ol; the estrogen anion is (9S,13S,14S,17S)-17-hydroxy-13-methyl-6,9,11,12,14,15,16,17-octahydrocyclopenta[a]phenanthrene-3-oxide, and the estrogen molecule is (9S,13 S,14S,17S)-13-methyl-6,9,11,12,14,15,16,17-octahydrocyclopenta[a]phenanthrene-3,17-diol; the estrogen anion is (9S,13S,14S,17R)-17-hydroxy-13-methyl-6,9,11,12,14,15,16,17-octahydrocyclopenta[a]phenanthrene-3-oxide, and the estrogen molecule is (9S,13S,14S,17R)-13-methyl-6,9,11,12,14,15,16,17-octahydrocyclopenta[a]phenanthrene-3,17-diol; the estrogen anion is (13 S,14 S,17 S)-17-hydroxy-13-methyl-6,7,11,12,14,15,16,17-octahydrocyclopenta[a]phenanthrene-3-oxide, and the estrogen molecule is (13S,14S,17S)-13-methyl-6,7,11,12,14,15,16,17-octahydrocyclopenta[a]phenanthrene-3,17-diol; the estrogen anion is (13S,14S,17R)-17-hydroxy-13-methyl-6,7,11,12,14,15,16,17-octahydrocyclopenta[a]phenanthrene-3-oxide, and the estrogen molecule is (13S,14S,17R)-13-methyl-6,7,11,12,14,15,16,17-octahydrocyclopenta[a]phenanthrene-3,17-diol; the estrogen anion is (13S,14S,17S)-17-hydroxy-13-methyl-11,12,14,15,16,17-hexahydrocyclopenta[a]phenanthrene-3-oxide, and the estrogen molecule is (13S,14S,17S) methyl-11,12,14,15,16,17-hexahydrocyclopenta[a]phenanthrene-3,17-diol; the estrogen anion is (13S,14S,17R)-17-hydroxy-13-methyl-11,12,14,15,16,17-hexahydrocyclopenta[a]phenanthrene oxide, and the estrogen molecule is (13S,14S,17R)-13-methyl-11,12,14,15,16,17-hexahydrocyclopenta[a]phenanthrene-3,17-diol; the estrogen anion is (8R,9S,13S,14S,15R,16R,17R)-15,16,17-trihydroxy-13-methyl-6,7,8,9,11,12,14,15,16,17-decahydrocyclopenta[a]phenanthrene-3-oxide, and the estrogen molecule is (8R,9S,13S,14S,15R,16R,17R)-13-methyl-6,7,8,9,11,12,14,15,16,17-decahydrocyclopenta[a]phenanthrene-3,15,16,17-tetrol; the estrogen anion is 4-[4-(4-hydroxyphenyl)hexa-2,4-dien-3-yl]phenolate, and the estrogen molecule is 4-[4-(4-hydroxyphenyl)hexa-2,4-dien-3-yl]phenol; the estrogen anion is 4-[4-(4-hydroxyphenyl)hex-3-en-3-yl]phenolate, and the estrogen molecule is 4-[4-(4-hydroxyphenyl)hex-3-en-3-yl]phenol; the estrogen anion is 4-[4-(4-methoxyphenyl)hex-3-en-3-yl]phenolate, and the estrogen molecule is 4-[4-(4-methoxyphenyl)hex-3-en-3-yl]phenol; the estrogen anion is 4-(4-{4-[(phenylmethyl)oxy]phenyl}hex-3-en-3-yl)phenolate, and the estrogen molecule is 4-(4-{4-[(phenylmethyl)oxy]phenyl}hex-3-en-3-yl)phenol; the estrogen anion is 4-[2-(4-hydroxyphenyl)vinyl]phenolate, and the estrogen molecule is 4-[2-(4-hydroxyphenyl)vinyl]phenol; the estrogen anion is 4-[3-(4-hydroxyphenyl)but-2-en-2-yl]phenolate, and the estrogen molecule is 4-[3-(4-hydroxyphenyl)but-2-en-2-yl]phenol; the estrogen anion is 4-[4-(4-hydroxyphenyl)hex-3-yl]phenolate, and the estrogen molecule is 4-[4-(4-hydroxyphenyl)hex-3-yl]phenol; the estrogen anion is 4-[3-ethyl-4-(4-hydroxyphenyl)hex-2-yl]phenolate, and the estrogen molecule is 4-[3-ethyl-4-(4-hydroxyphenyl)hex-2-yl]phenol; the estrogen anion is 4-[4-ethyl-5-(4-hydroxyphenyl)hex-3-yl]phenolate, and the estrogen molecule is 4-[4-ethyl-5-(4-hydroxyphenyl)hex-3-yl]phenol; or the estrogen anion is 4-[4-(4-hydroxy-3-methylphenyl)hex-3-yl]-2-methylphenolate; and the estrogen molecule is 4-[4-(4-hydroxy-3-methylphenyl)hex-3-yl]-2-methylphenol. 28. The method of claim 25 or 26, wherein: the estrogen anion is (8R,9S,13S,14S,17S)-17-hydroxy-13-methyl-6,7,8,9,11,12,14,15,16,17-decahydrocyclopenta[a]phenanthrene-3-oxide, and the estrogen molecule is (8R,9S,13S,14S,17S)-13-methyl-6,7,8,9,11,12,14,15,16,17-decahydrocyclopenta[a]phenanthrene-3,17-diol; the estrogen anion is (8R,9S,13S,14S,17R)-17-ethynyl-17-hydroxy-13-methyl-7,8,9,11,12,14,15,16-octahydro-6H-cyclopenta[a]phenanthrene-3-oxide, and the estrogen molecule is (8R,9S,13S,14S,17R) ethynyl-13-methyl-7,8,9,11,12,14,15,16-octahydro-6H-cyclopenta[a]phenanthrene-3,17-diol; or the estrogen anion is (8R,9S,13S,14S,16R,17R)-16,17-dihydroxy-13-methyl-6,7,8,9,11,12,14,15,16,17-decahydrocyclopenta[a]phenanthrene-3-oxide, and the estrogen molecule is (8R,9S,13 S,14S,16R,17R)-13-methyl-6,7,8,9,11,12,14,15,16,17-decahydrocyclopenta[a]phenanthrene-3,16,17-triol.

In some embodiments, the composition comprises the estrogen molecule; and the composition comprises the estrogen anion at a greater molar concentration than the estrogen molecule when the composition is provided.

In some embodiments, the method comprises converting the estrogen anion into the estrogen molecule in situ subsequent to administering the composition to the subject.

In some embodiments, the method comprises converting the estrogen anion into the estrogen molecule ex vivo prior to administering the composition to the subject.

In some embodiments, the amount is effective either as a contraceptive, to treat symptoms of menstruation, to treat dysmenorrhea, to treat menorrhagia, to treat polycystic ovary syndrome, to treat endometriosis, to treat female hypogonadism, to treat absence of menstruation, to treat symptoms of menopause, to provide perimenopausal or postmenopausal hormone replacement therapy, to provide feminizing hormone therapy, to treat hirsutism, to treat acne, or two or more of the foregoing. In some embodiments, the amount is effective to treat a psychiatric condition or a neurodegenerative disease. In some embodiments, the amount is effective to treat anxiety, schizophreniform disorder, schizophrenia, multiple sclerosis, mild cognitive impairment, or Alzheimer's disease. In some embodiments, the amount is effective to treat inflammation, an autoimmune disease, or sepsis. In some embodiments, the amount is effective to treat arthritis, ankylosing spondylitis, an inflammatory autoimmune-mediated arthritis, rheumatoid arthritis, psoriatic arthritis, psoriasis, plaque psoriasis, lupus, Sjogren's syndrome, inflammatory bowel disease, Crohn's disease, or ulcerative colitis. In some embodiments, the amount is effective to treat breast cancer, prostate cancer, or benign prostatic hyperplasia.

In some embodiments, the composition is a liquid, and the estrogen anion is dissolved in the liquid.

Various aspects of this patent document relate to a method to administer a cannabinoid to a subject, comprising providing a composition comprising a cannabinoid anion and administering the composition to the subject.

In some embodiments, the cannabinoid anion is 3-hydroxy-2-[(1R,6R)-6-isopropenyl methylcyclohex-2-enyl]-5-pentylphenolate; 3-hydroxy-2-[(1R,6R)-6-isopropenyl methylcyclohex-2-enyl]-5-propylphenolate; 2-geranyl-3-hydroxy-5-pentylphenolate; 2-geranyl hydroxy-5-propylphenolate; (6aR,10aR)-6,6,9-trimethyl-3-pentyl-6a,7,8,10a-tetrahydro-6H-benzo[c]chromene-1-oxide; (6aR,10aR)-6,6,9-trimethyl-3-propyl-6a,7,8,10a-tetrahydro-6H-benzo[c]chromene-1-oxide; 6,6,9-trimethyl-3-pentyl-6H-benzo[c]chromene-1-oxide; or 6,6,9-trimethyl-3-propyl-6H-benzo[c]chromene-1-oxide.

In some embodiments, the method comprises contacting the cannabinoid anion with a Brønsted acid to convert the cannabinoid anion into a cannabinoid molecule, wherein the cannabinoid molecule has an acid dissociation constant in water of at least 50 femtomolar and no greater than 50 nanomolar for conversion of the cannabinoid molecule into the cannabinoid anion.

In some embodiments, the administering is either oral or topical administering.

Various aspects of this patent document relate to a method to administer a cannabinoid to a subject, comprising providing a composition comprising a cannabinoid anion and orally administering the composition to the subject, wherein: the cannabinoid anion has a conjugate acid that is a cannabinoid molecule; the cannabinoid molecule has an acid dissociation constant in water of at least 50 femtomolar and no greater than 50 nanomolar for conversion of the cannabinoid molecule into the cannabinoid anion; the composition is formulated to allow the conversion of the cannabinoid anion into the cannabinoid molecule before the cannabinoid anion reaches the stomach of the subject; and the composition is formulated to allow absorption of the cannabinoid molecule by the epithelial lining of the gastrointestinal tract between the lips and the stomach, excluding the stomach and the outer surfaces of the lips, and including the esophagus and the inner surfaces of the lips.

In some embodiments, the composition comprises the cannabinoid molecule; and the composition comprises the cannabinoid anion at a greater molar concentration than the cannabinoid molecule when the composition is provided.

In some embodiments, the method comprises converting the cannabinoid anion into the cannabinoid molecule in situ subsequent to administering the composition to the subject.

In some embodiments, the method comprises converting the cannabinoid anion into the cannabinoid molecule ex vivo prior to administering the composition to the subject.

In some embodiments, the cannabinoid anion is 3-hydroxy-2-[(1R,6R)-6-isopropenyl-3-methylcyclohex-2-enyl]-5-pentylphenolate, and the cannabinoid molecule is 2-[(1R,6R) isopropenyl-3-methylcyclohex-2-enyl]-5-pentylbenzene-1,3-diol; the cannabinoid anion is 3-hydroxy-2-[(1R,6R)-6-isopropenyl-3-methylcyclohex-2-enyl]-5-propylphenolate, and the cannabinoid molecule is 2-[(1R,6R)-6-isopropenyl-3-methylcyclohex-2-enyl]-5-propylbenzene-1,3-diol; the cannabinoid anion is 2-geranyl-3-hydroxy-5-pentylphenolate, and the cannabinoid molecule is 2-geranyl-5-pentylbenzene-1,3-diol; the cannabinoid anion is 2-geranyl-3-hydroxy propylphenolate, and the cannabinoid molecule is 2-geranyl-5-propylbenzene-1,3-diol; the cannabinoid anion is (6 aR, 10 aR)-6,6,9-trimethyl-3-pentyl-6a,7,8,10a-tetrahydro-6H-benzo[c]chromene-1-oxide, and the cannabinoid molecule is (6aR,10aR)-6,6,9-trimethyl-3-pentyl-6a,7,8,10a-tetrahydro-6H-benzo[c]chromene-1-ol; the cannabinoid anion is (6aR,10aR)-6,6,9-trimethyl-3-propyl-6a,7,8,10a-tetrahydro-6H-benzo[c]chromene-1-oxide, and the cannabinoid molecule is (6 aR,10 aR)-6,6,9-trimethyl-3-propyl-6a,7,8,10a-tetrahydro-6H-benzo[c]chromene-1-ol; the cannabinoid anion is 6,6,9-trimethyl-3-pentyl-6H-benzo[c]chromene-1-oxide, and the cannabinoid molecule is 6,6,9-trimethyl-3-pentyl-6H-benzo[c]chromene-1-ol; or the cannabinoid anion is 6,6,9-trimethyl-3-propyl-6H-benzo[c]chromene-1-oxide, and the cannabinoid molecule is 6,6,9-trimethyl-3-propyl-6H-benzo[c]chromene-1-ol.

In some embodiments, the cannabinoid anion is 3-hydroxy-2-[(1R,6R)-6-isopropenyl-3-methylcyclohex-2-enyl]-5-pentylphenolate or 3-hydroxy-2-[(1R,6R)-6-isopropenyl-3-methylcyclohex-2-enyl]-5-propylphenolate.

In some embodiments, the composition is a liquid, and the cannabinoid anion is dissolved in the liquid.

In some embodiments, the amount is effective to prophylactically prevent or treat muscle cramping, muscle spasms, restless-legs syndrome, nystagmus, a dyskinetic movement disorder, tremor, seizures, epilepsy, muscular dystrophy, or inclusion body myositis.

In some embodiments, the method comprises administering the composition to the subject while the subject is having an active seizure, wherein the composition is effective to either arrest or reduce the severity of the active seizure.

In some embodiments, the amount is effective to reduce blood pressure in the subject. In some embodiments, the amount is effective to prophylactically prevent or treat prehypertension or hypertension. In some embodiments, the amount is effective to treat attention deficit hyperactivity disorder (“ADHD”), autism or an autism spectrum disorder, Asperger syndrome, fragile X syndrome, Down syndrome, a pervasive developmental disorder not otherwise specified (“PDD-NOS”), a childhood disintegrative disorder, or Tourette's syndrome. In some embodiments, the amount is effective to treat anxiety, post-traumatic stress disorder (“PTSD”), depression, bipolar disorder, obsessive-compulsive disorder, schizophreniform disorder, schizophrenia, or psychosis. In some embodiments, the amount is effective to treat pain or inflammation. In some embodiments, the amount is effective to treat an autoimmune disorder. In some embodiments, the amount is effective to treat arthritis, ankylosing spondylitis, an inflammatory autoimmune-mediated arthritis, rheumatoid arthritis, psoriatic arthritis, psoriasis, plaque psoriasis, lupus, Sjogren's syndrome, inflammatory bowel disease, Crohn's disease, or ulcerative colitis. In some embodiments, the amount is effective to treat a neurodegenerative disease. In some embodiments, the amount is effective to treat Parkinson's Disease, and treating the Parkinson's Disease comprises treating Parkinsonian tremor. In some embodiments, the amount is effective to treat multiple sclerosis, mild cognitive impairment, Alzheimer's Disease, amyotrophic lateral sclerosis (“ALS”), or Huntington's disease. In some embodiments, the amount is effective to treat obesity, metabolic syndrome, or diabetes mellitus. In some embodiments, the amount is effective to treat a viral infection or a bacterial infection. In some embodiments, the amount is effective to treat an infection caused by Escherichia coli, Klebsiella pneumoniae, Pseudomonas aeruginosa, Acinetobacter baumannii, Moraxella catarrhalis, Legionella pneumophila, Staphylococcus aureus, Streptococcus pneumoniae, Enterococcus faecium, Clostridioides difficile, Mycobacterium tuberculosis, Neisseria gonorrhoeae, Cutibacterium acnes, or COVID-19.

Various aspects of this patent document relate to a method to prophylactically prevent or treat seizures in a subject, comprising providing a composition comprising 3-hydroxy-2-[(1R,6R)-6-isopropenyl-3-methylcyclohex-2-enyl]-5-pentylphenolate or 3-hydroxy-2-[(1R,6R)-6-isopropenyl-3-methylcyclohex-2-enyl]-5-propylphenolate, and administering the composition to the subject in an amount that is effective to prophylactically prevent or treat seizures.

Various aspects of this patent document relate to a method to administer a substituted phenol to a subject, comprising providing a composition comprising a substituted phenolate and orally administering the composition to the subject, wherein: the substituted phenolate has a conjugate acid that is the substituted phenol; the substituted phenol has an acid dissociation constant in water of at least 50 femtomolar and no greater than 50 nanomolar for conversion of the substituted phenol into the substituted phenolate; the composition is formulated to allow the conversion of the substituted phenolate into the substituted phenol before the substituted phenolate reaches the stomach of the subject; and the composition is formulated to allow absorption of the substituted phenol by the epithelial lining of the gastrointestinal tract between the lips and the stomach, excluding the stomach and the outer surfaces of the lips, and including the esophagus and the inner surfaces of the lips.

In some embodiments, the method comprises converting the substituted phenolate into the substituted phenol in situ subsequent to administering the composition to the subject.

In some embodiments, the method comprises converting the substituted phenolate into the substituted phenol ex vivo prior to administering the composition to the subject.

In some embodiments, the composition comprises the substituted phenol, and the composition comprises the substituted phenolate at a greater molar concentration than the substituted phenol when the composition is provided.

In some embodiments, the substituted phenol is 2-methoxy-4-(prop-2-enyl)phenol, and the substituted phenolate is 2-methoxy-4-(prop-2-enyl)phenolate. In some embodiments, the substituted phenol is 5-methyl-2-(prop-2-yl)phenol, and the substituted phenolate is 5-methyl-2-(prop-2-yl)phenolate. In some embodiments, the substituted phenol is 2-methyl-5-(prop-2-yl)phenol, and the substituted phenolate is 2-methyl-5-(prop-2-yl)phenolate. In some embodiments, the substituted phenol is 2-methoxy-4-({N-[(6E)-1-oxo-8-methylnon-6-enyl]amino}methyl)phenol, and the substituted phenolate is 2-methoxy-4-({N-[(6E)-1-oxo-8-methylnon-6-enyl]amino}methyl)phenolate. In some embodiments, the substituted phenol is 2-methoxy-4-[(5S)-3-oxo-5-hydroxydecanyl]phenol, and the substituted phenolate is 2-methoxy-4-[(5S)-3-oxo-5-hydroxydecanyl]phenolate. In some embodiments, the substituted phenol is 4-formyl-2-methoxyphenol, and the substituted phenolate is 4-formyl-2-methoxyphenolate. In some embodiments, the substituted phenol is 4-formyl-2-ethoxyphenol, and the substituted phenolate is 4-formyl-2-ethoxyphenolate. In some embodiments, the substituted phenol is 4-(3-oxobutyl)phenol, and the substituted phenolate is 4-(3-oxobutyl)phenolate. In some embodiments, the substituted phenol is 2-methoxy-4-[3,5-dioxo-7-(4-hydroxy-3-methoxyphenyl)hepta-1,6-dieneyl]phenol, and the substituted phenolate is 2-methoxy-4-[3,5-dioxo-7-(4-hydroxy-3-methoxyphenyl)hepta-1,6-dieneyl]phenolate. In some embodiments, the substituted phenol is 2,6-diisopropylphenol, and the substituted phenolate is 2,6-diisopropylphenolate.

Various aspects of this patent document relate to a method to administer a molecule to a subject, comprising providing a composition comprising an anion; converting the anion into the molecule; and administering the composition to the subject, wherein the molecule has an acid dissociation constant in water of at least 50 femtomolar and no greater than 50 nanomolar for conversion of the molecule into the anion.

In some embodiments, the anion is dissolved in the composition at a concentration that is greater than the solubility of the molecule in water.

In some embodiments, the molecule has an octanol-water partition coefficient that is greater than 1. In some specific embodiments, the molecule has an octanol-water partition coefficient that is greater than 3. In some very specific embodiments, the molecule has an octanol-water partition coefficient that is greater than 5.

In some embodiments, the administering is orally administering; the composition is formulated to allow the conversion of the anion into the molecule before the anion reaches the stomach of the subject; and the composition is formulated to allow absorption of the molecule by the epithelial lining of the gastrointestinal tract between the lips and the stomach, excluding the stomach and the outer surfaces of the lips, and including the esophagus and the inner surfaces of the lips.

In some embodiments, the method comprises converting the anion into the molecule in situ subsequent to administering the composition to the subject.

In some embodiments, the method comprises converting the anion into the molecule ex vivo prior to administering the composition to the subject.

In some embodiments, the composition comprises the molecule, and the composition comprises the anion at a greater molar concentration than the molecule when the composition is provided.

In some embodiments, the anion has a general structure Ia, Ib, or Ic.

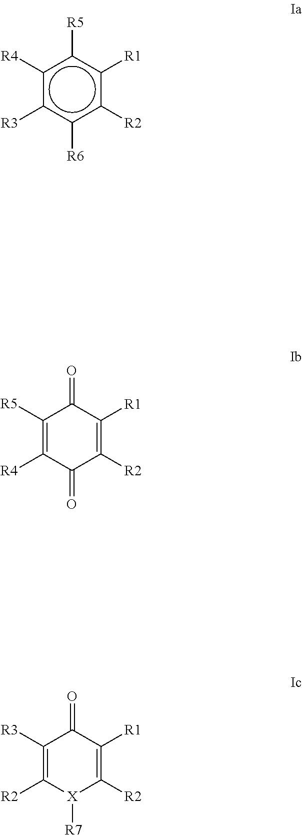

In some embodiments, (i) one of R1, R2, R3, and R4 is oxide; (ii) one of R1, R2, R3, R4, R5 and R6 is selected from hydro; hydroxy; methoxy; fluoro; chloro; bromo; and iodo; (iii) one of R1, R2, R3, R4, R5 and R6 is selected from hydro; hydroxy; hydroxymethyl; 2-hydroxyethyl; 1,2-dihydroxyethyl; 3-hydroxyprop-1-enyl; methyl; 2-methylprop-2-yl; methoxy; ethoxy; propoxy; butoxy; pentoxy; hexoxy; heptoxy; octanoxy; (prop-2-yl)oxy; isoprenyloxy; benzyloxy; [4-(prop-2-enyl)phenyl]oxy; fluoro; chloro; bromo; iodo; amino; and nitro; (iv) one of R1, R2, R3, R4, R5 and R6 is selected from hydro; hydroxy; methoxy; formyl; acetyl; 2-oxoethyl; 1-oxopropyl; 1-oxobutyl; (prop-2-yl)carbonyl; 3-oxobutyl; 3-oxobut-1-enyl; (methoxy)carbonyl; (ethoxy)carbonyl; (propoxy)carbonyl; (2-propoxy)carbonyl; (butoxy)carbonyl; (pentoxy)carbonyl; (hexoxy)carbonyl; and (heptoxy)carbonyl; (v) one of R1, R2, R3, R4, R5 and R6 is selected from hydro; methoxy; methyl; ethyl; propyl; butyl; pentyl; hexyl; heptyl; octanyl; nonanyl; decanyl; undecanyl; dodecanyl; tridecyl; tetradecyl; pentadecyl; prop-2-yl; but-2-yl; pent-2-yl; hex-2-yl; hept-2-yl; octan-2-yl; nonan-2-yl; decan-2-yl; 2-methylprop-2-yl; 2-methylbut-2-yl; 2-methylpent-2-yl; 2-methylhex-2-yl; 2-methylhept-2-yl; 2-methyloctan-2-yl; 2-methylnonan-2-yl; 2-methyldecan-2-yl; 3-methylbut-2-yl; 3-methylpent-2-yl; 3-methylhex-2-yl; 3-methylhept-2-yl; 3-methyloctan-2-yl; 3-methylnonan-2-yl; 3-methyldecan-2-yl; 2,4,4-trimethylpent-2-yl; vinyl; prop-1-enyl; prop-2-enyl; pentadec-8-enyl; 2-methylbut-1-en-3-yl; 3-methylbut-1-en-3-yl; 3-ethenyl-3,7-dimethylocta-1,6-dienyl; 4-ethenyl-4,7-dimethylocta-1,6-dien-2-yl; isoprenyl; geranyl; 3,7,11,15,19,23,27,31,35,39-decamethyltetraconta-2,6,10,14,18,22,26,30,34,38-decaenyl; phenyl; benzyl; and 2-phenylethyl; and (vi) one of R1, R2, R3, R4, R5 and R6 is selected from hydro; methyl; ethyl; propyl; 2-methylprop-2-yl; 2-methylbut-2-yl; isoprenyl; geranyl; 6-methylhept-5-en-2-yl; 6-methoxy-5-[(methoxy)carbonyl]-4-methylhexa-1,3,5-trienyl; 2-{[1-oxo-4-formyl-3-(2-oxoethyl)hex-4-enyl]oxy}ethyl; 3-oxobutyl; 3-oxobut-1-enyl; 3-oxooct-4-enyl; 3-oxodec-4-enyl; 3-oxododec-4-enyl; 3-oxotetradec-4-enyl; 3-oxohexadec-4-enyl; 3-oxo-5-hydroxyoctanyl; 3-oxo-5-hydroxydecanyl; 3-oxo-5-hydroxydodecanyl; 3-oxo-5-hydroxytetradecanyl; 3-oxo-5-hydroxyhexadecanyl; [N-(1-oxononanyl)amino]methyl; {N-[1-oxo-12-(1-oxo-2-phenylethyl)octadec-9-enyl]amino}methyl; [N-(1-oxo-8-methylnon-6-enyl)amino]methyl; [N-(1-oxo-8-methylnonanyl)amino]methyl; [N-(1-oxo-7-methyloctanyl)amino]methyl; [N-(1-oxo-9-methyldec-6-enyl)amino]methyl; [N-(1-oxo-9-methyldecanyl)amino]methyl; [N-(1-oxo-8-methyldec-6-enyl)amino]methyl; 2-{-[4-(4-hydroxyphenyl)but-2-yl]amino}ethyl; 3-{[2-(3,4-dihydroxyphenyl)ethyl]amino}butyl; 1-hydroxy-2-{[1-(4-hydroxyphenyl)prop-2-yl]amino}ethyl; 2-[2-hydroxy-2-(3,5-dihydroxyphenyl)ethyl]amino propyl; 4-{[1-oxo-3,7-dimethyl-9-(2,6,6-trimethylcyclohex-1-enyl)non-2,4,6,8-tetraenyl]amino; cyclohexyl; cycloheptyl; adamant-1-yl; adamant-2-yl; 3,3-dimethylcycolhexyl; 6,6-dimethyl-4-oxo-2-bicyclo[3.1.1]heptanyl; 3-hydroxycyclohexyl; 5-hydroxy-2-(3-hydroxypropyl)cyclohexyl; 6-(prop-1-en-2-yl)-3-methylcyclohex-2-enyl; 6-(prop-2-yl)-3-methylcyclohex-2-enyl; 6-(prop-1-en-2-yl)-3-methylcyclohex-3-enyl; 6-(prop-2-yl)-3-methylcyclohex-3-enyl; 4-hydroxy-3-(prop-2-enyl)phenyl; 6-hydroxy-3-(prop-2-enyl)phenyl; 4-methoxy-3-(prop-2-enyl)phenyl; (4-hydroxyphenyl)methyl; 2-(4-hydroxyphenyl)ethyl; 2-(3,5-dihydroxyphenyl)ethyl; 2-hydroxy-2-(3,4,5-trimethoxyphenyl)ethyl; 2-phenylethenyl; 2-(4-hydroxyphenyl)ethenyl; 2-(3,4-dihydroxyphenyl)ethenyl; 2-(3,5-dihydroxyphenyl)ethenyl; 2-(3,4,5-trimethoxyphenyl)ethenyl; 2-(4-hydroxyphenyl)prop-2-yl; 1-oxo-3-phenylpropyl; 1-oxo-3-(4-hydroxyphenyl)propyl; 3-oxo (2,4,6-trihydroxyphenyl)propyl; 1-oxo-3-phenylprop-2-enyl; 3-oxo-3-(4-hydroxyphenyl)prop enyl; 1-oxo-3-(4-hydroxyphenyl)prop-2-enyl; 3-oxo-3-(2,4-dihydroxyphenyl)prop-1-enyl; 1-oxo (3,4-dihydroxy-2-methoxyphenyl)prop-2-enyl; 1-oxo-3-[4-hydroxy-3,5-bis(isoprenyl)phenyl]prop-2-enyl; 3-oxo-3-[3,5-dihydroxy-4-isoprenylphenyl]prop-1-enyl; 3-oxo-3-[4,6-dihydroxy methoxy-5-isoprenylphenyl]prop-1-enyl; 2-(4-hydroxyphenyl)but-2-yl; 2,3-dimethyl-4-(3,4-dihydroxyphenyl)butyl; 2,3-dimethyl-4-(4-hydroxy-3-methoxyphenyl)butyl; 2,3-bis(hydroxymethyl)-4-(3-hydroxyphenyl)butyl; 2,3-bis(hydroxymethyl)-4-(4-hydroxy-3-methoxyphenyl)butyl; 3-(4-hydroxyphenyl)but-2-en-2-yl; 4-(4-hydroxyphenyl)hex-3-yl; 4-(4-hydroxy-3-methylphenyl)hex-3-yl; 3-ethyl-4-(4-hydroxyphenyl)hex-2-yl; 4-ethyl-5-(4-hydroxyphenyl)hex-3-yl; 4-(4-hydroxyphenyl)hex-3-en-3-yl; 3-(4-hydroxyphenyl)-2-methylpent-1-enyl; 1-(4-hydroxyphenyl)-2-methylpent-1-en-3-yl; 4-(4-methoxyphenyl)hex-3-en-3-yl; 4-{4-[(phenylmethyl)oxy]phenyl}hex-3-en-3-yl; 4-(4-hydroxyphenyl)hexa-2,4-dien-3-yl; 3-oxo-7-(4-hydroxyphenyl)hepta-1,4,6-trieneyl; 3-oxo-7-(4-hydroxyphenyl)hepta-1,3,6-trieneyl; 7-(4-hydroxyphenyl)-3,5-dioxohepta-1,6-dieneyl; 7-(4-hydroxy-3-methoxyphenyl)-3,5-dioxohepta-1,6-dieneyl; [6-oxo-2,4-dihydroxy-3,3-dimethyl-5-(1-oxo-2-methylpropyl)cyclohexa-1,4-dienyl]methyl; [2,6-dihydroxy-4-methoxy-5-methyl-3-(1-oxo-2-methylpropyl)phenyl]methyl; [6-hydroxy-2,4-dimethoxy-5-methyl-3-(1-oxo-2-methylpropyl)phenyl]methyl; 1-oxo-3-[4-hydroxy-2-methoxy-3-isoprenylphenyl]prop-2-enyl; 1-oxo-3-[4-hydroxy-2-methoxy-3-(2-methylbut-1-en-3-yl)phenyl]prop-2-enyl; 1-oxo-3-[4-hydroxy-2-methoxy-5-(2-methylbut-1-en-3-yl)phenyl]prop-2-enyl; 1-oxo-3-[4-hydroxy-2-methoxy-5-(3-methylbut-1-en-3-yl)phenyl]prop-2-enyl; 1-oxo-3-[4-hydroxy-3,5-bis(isoprenyl)phenyl]prop-2-enyl; phenylcarbonyl; (4-methylphenyl)carbonyl; (2-hydroxyphenyl)carbonyl; (2,4-dihydroxyphenyl)carbonyl; (2-hydroxy-4-methoxyphenyl)carbonyl; [(3,3,5-trimethylcyclohexyl)oxy]carbonyl; [(3-hydroxy-4-methoxycarbonyl-2,5-dimethylphenyl)oxy]carbonyl; [(3-formyl-2,4-dihydroxy-6-methylphenyl)carbonyl]oxy; (4-hydroxyphenyl)-[2-(hydroxymethyl)phenyl]methyl; 1,7,7-trimethyl-2-bicyclo[2.2.1]heptyl; {4-oxo-5-[(3-hydroxyphenyl)methyl]-3-oxacyclopentyl}methyl; {2-oxo-5-[(3-hydroxyphenyl)methyl]-3-oxacyclopentyl}methyl; {2-oxo-3-[(3-methyoxy-4-hydroxyphenyl)methylidene]cyclohexylidene)methyl; 2-(4-methoxy-2-oxo-2H-pyran-6-yl)ethenyl; 4-[(4-hydroxy-3-methoxyphenyl)methyl]-3-(hydroxymethyl)tetrahydrofuran-2-yl; [2-(4-hydroxy-3-methoxyphenyl)-3-(hydroxymethyl)tetrahydrofuran-4-yl]methyl; {2-oxo-4-[(3,4-dimethoxyphenyl)methyl]tetrahydrofuran-3-yl}methyl; {2-oxo-4-[(3-methoxy hydroxyphenyl)methyl]tetrahydrofuran-3-yl}methyl; {2-oxo-3-[(3-methoxy hydroxyphenyl)methyl]tetrahydrofuran-4-yl}methyl; {2-oxo-4-[(3-methoxy hydroxyphenyl)hydroxymethyl]tetrahydrofuran-3-yl}methyl; {2-oxo-3-[(3-methoxy hydroxyphenyl)methyl]tetrahydrofuran-4-yl}hydroxymethyl; 7-[5,5-dimethyl-4-oxo-tetrahydrofuran-2-yl]-3-methylocta-2,6-dienyl; (1,2,4a-trimethyl-5-methylidene-3,4,6,7,8,8a-hexahydro-2H-naphthalen-1-yl)methyl; 3-hydroxy-7-hydroxymethyl-1,2,3,4,4a,5,6,7,8,8a-decahydronaphthalen-1-yl; 6-(4-hydroxy-3-methoxyphenyl)-1,3,3a,4,6,6a-hexahydrofuro[3,4-c]furan-3-yl; 6-(4-hydroxy-3,5-dimethoxyphenyl)-1,3,3a,4,6,6a-hexahydrofuro[3,4-c]furan-3-yl; 7-oxo-4-hydroxy-5-methyl-5,6-dihydro-4H-2-benzofuran-1-yl; 3-hydroxymethyl-7-methoxy-5-(3-oxoprop-1-enyl)-2,3-dihydro-1-benzofuran-2-yl; (2-ethyl-1-benzofuran-3-yl)carbonyl; (3-oxo-4-hydroxy-2-benzofuran-1-ylidene)methyl; (3-oxo-6-hydroxy-1-benzofuran-2-ylidene)methyl; (3-oxo-4,6-dihydroxy-1-benzofuran-2-ylidene)methyl; (3-oxo-6-hydroxy-4-methoxy-1-benzofuran-2-ylidene)methyl; 4-(1,3-benzodioxol-5-yl)-2,3-dimethylbutyl; 8-oxo-4-hydroxy-5a,6,8a,9-tetrahydro-5H-[2]benzofuro[5,6-f][1,3]benzodioxol-9-yl; 7-hydroxy-2H-chromen-3-yl; 4-oxo-7-methoxy-4H-chromen-2-yl; 1-oxo-8-hydroxy-3,4-dihydro-1H-isochromen-3-yl; (3,4,7-trihydroxy-3,4-dihydro-2H-chromen-3-yl)methyl; (4-oxo-5-hydroxy-6,7-dimethoxy-2,3-dihydro-4H-chromen-3-yl)methyl; [2-oxo-5-hydroxy-7-(2-methylnonan-2-yl)-2H-chromen-3-yl]methyl; 5,7-dihydroxy-8-(1-oxo-3-phenylprop-2-enyl)-2,2-dimethyl-2H-chromen-6-yl]methyl; 5,7-dimethoxy-8-(1-oxo-3-phenylprop-2-enyl)-2,2-dimethyl-2H-chromen-6-yl]methyl; 5,7-dihydroxy-8-(1-oxo-3-phenylprop-2-enyl)-2,2-dimethyl-3,4-dihydro-2H-chromen-6-yl; 4-oxo-5,7-dihydroxy-8-[2-hydroxy-5-(4-oxo-5,7-dihydroxy-4H-chromen-2-yl)phenyl]-4H-chromen-2-yl; 4-oxo-8-[6-(2,4-dihydroxybenzoyl)-5-(2,4-dihydroxyphenyl)-3-methylcyclohex-2-enyl]-5,7-dihydroxy-3 isoprenyl-4H-chromen-2-yl; 8-oxo-2-hydroxymethyl-5-methoxy-2,3-dihydropyrano[2,3-h][1,4]benzodioxin-3-yl; 9-oxo-5-methoxy-2-{[(acetyl)oxy]methyl}-2,3-dihydropyrano[3,2-h][1,4]benzodioxin-3-yl; 2-hydroxymethyl-7-(4-oxo-3,5,7-trihydroxy-2,3-dihydro-4H-chromen-2-yl)-2,3-dihydro-1,4-benzodioxin-3-yl; 8-oxo-9-hydroxy-[1,3]diolxolo[4,5-g]-4H-chromen-7-yl; 8-oxo-9-methoxy-[1,3]diolxolo[4,5-g]-4H-chromen-7-yl; 8,8-dimethyl-3,4-dihydro-2H-pyrano[2,3-f]chromen-3-yl; 4-oxo-5-hydroxy-6-isoprenyl-8,8-dimethylpyrano[2,3-h]-4H-chromen-3-yl; 7-hydroxy-3-hydroxymethyl-4-(4-oxo-3,5,7-trihydroxy-2,3-dihydro-4H-chromen-2-yl)-2,3-dihydro-1-benzofuran-2-yl; 7-hydroxy-3-hydroxymethyl-5-(4-oxo-3,5,7-trihydroxy-2,3-dihydro-4H-chromen-2-yl)-2,3-dihydro-1-benzofuran-2-yl; 2-oxo-3-hydroxy-8-(3,5,7-trihydroxy-4-oxo-2,3-dihydro-4H-chromen-2-yl)-4-oxatricyclo[4.3.1.0^{3,7}]dec-8-en-10-yl; 6,9,17,19,21-pentahydroxy-5-(4-hydroxyphenyl)-4,12,14-trioxapentacyclo[11.7.1. 0^{2,11}. 0.0^{3,8}. 0.0^{15,20}]henicosa-2(11),3(8),9,15,17,19-hexaen-13-yl; 6,9,17,19,21-pentahydroxy-3-(4-hydroxyphenyl)-4,12,14-trioxapentacyclo[11.7.1.0^{2,11}.0^{3,8}.0^{15,20}]henicosa-2(11),3(8),9,15,17,19-hexaen-5-yl; N-(4-phenyl)carbamoyl; N-(4-hydroxyphenyl)carbamoyl; [(2-hydroxyphenyl)carbonyl]amino; 1-oxo-icosa-5,8,11,14-tetraenyl)amino; N-(4-chlorophenyl)carbamoyl; 1-oxo-2-(2,6-dioxopiperidin yl)ethyl; 1-hydroxy-2-{[4-(4-hydroxyphenyl)butyl]amino}ethyl; 4-{[2-hydroxy-2-(3,4-dihydroxyphenyl)ethyl]amino}butyl; [(4-oxo-1,4-dihydroquinolin-3-yl)carbonyl]amino; 3,5-dioxo-4-butyl-2-phenylpyrazolidin-1-yl; (2-{[4-hydroxy-3,5-di(2-methylprop-2-yl)phenyl]sulfanyl}propan-2-yl)sulfanyl; (3-chloro-6-hydroxyphenyl)methyl; (2,4-dichlorophenyl)methyl; 3-(3-fluoro-4-hydroxyphenyl)pent-2-yl; and 2-(3-fluoro-4-hydroxyphenyl)pent-3-yl. In the case of general structures Ib and Ic, which each lack R3 and R6, one of R1, R2, R4, and R5 is selected from group (i) oxide, and the other three of R1, R2, R4, and R5 are each selected from group (ii), (iii), (iv), (v), and (vi) such that each of R1, R2, R4, and R5 is selected from a different group, and none of R1, R2, R4, and R5 are selected from two of groups (ii), (iii), (iv), (v), and (vi). In some embodiments, X is selected from 0 and N, wherein when X is 0, then R7 is absent; and when X is N, then R7 is selected from hydro and methyl. In some embodiments, the anion has a molecular weight that is greater than 108 grams per mole. In some specific embodiments, the anion has a general structure Ia; R1 is oxide; R2 is hydro, methyl, 2-methylprop-2-yl, geranyl, hydroxy, methoxy, ethoxy, hydroxymethyl, formyl, or amino; and each of R3, R5, and R6 is hydro. In some specific embodiments, the anion has a general structure Ia; R1 is oxide; and at least 4 of R2, R3, R4, R5, and R6 are selected from hydro, hydroxy, and methoxy. In some very specific embodiments, the anion has a general structure Ia, Ib, or Ic, and the molecule is abnormal cannabidiol; acetosyringone; actiphenol; adipostatin A; aleuritin; alpha-kosin; alpha-peltatin; AM404; amentoflavone; amylmetacresol; apocynin; arbutamine; arctigenin; ascofuranone; aspidinol; atranorin; aureusidin; bakuchiol; balanophonin; benzarone; benzbromarone; benzestrol; benziodarone; benzophenone-2; benzophenone-6; benzoresorcinol; beta-kosin; beta-resorcylaldehyde; bifluranol; bilobol; bisdemethoxycurcumin; bisphenol A; bisphenol B; bisphenol F; bromosalicylchloranilide; bromosaligenin; butylated hydroxyanisole; butylated hydroxytoluene; butylparaben; cannabicyclohexanol; cannabidiol; cannabidiphorol; cannabidivarin; cannabigerol; cannabigerovarin; canolol; capsaicin; carvacrol; chavibetol; chavicol; clofoctol; clorophene; combretastatin; combretastatin A-1; combretastatin A-4; combretastatin B-1; coniferyl alcohol; cotoin; CP 55,244; CP 55,940; (C6)-CP 47,497; (C7)-CP 47,497; (C9)-CP 47,497; curcumin; cyclovalone; DB-2073; deferiprone; dehydroequol; demethoxycurcumin; dianol; dichlorophen; dienestrol; diethylstilbestrol; diethylstilbestrol monobenzyl ether; dihydrocapsaicin; dihydrokanakugiol; dihydroresveratrol; dimethylheptyl cannabidiol; dimethylstilbestrol; dioxybenzone; dobutamine; DOPAL; DOPEG; drupanol; durantin A; embelin; enterodiol; enterolactone; ethyl maltol; ethyl vanillin; ethylparaben; eugenol; fenoterol; fenretinide; flopropione; fumigatin; gentisyl alcohol; geranin A; geranylhydroquinone; [6]-gingerol; glabridin; guaiacol; heminordihydroguaiaretic acid; heptylparaben; hexestrol; homocapsaicin I; homocapsaicin II; homodihydrocapsaicin; homosalate; homovanillyl alcohol; honokiol; HU-331; hydroxymatairesinol; hydroxytyrosol; ilimaquinone; irilone; irisolone; isoeugenol; isoliquiritigenin; isosilybin A; isosilybin B; isosilychristin; ivacaftor; kanakugiol; kuwanon G; lariciresinol; leptosidin; leptosphaerin A; leptosphaerin B; licochalcone A; licochalcone B; licochalcone C; licochalcone D; licochalcone E; licochalcone F; macelignan; magnolol; maltol; matairesinol; mequinol; mestilbol; meta-cresol; methestrol; methylparaben; mexenone; monobenzone; nonivamide; nordihydrocapsaicin; nordihydroguaiaretic acid; 0-1602; 0-1871; obovatol; octabenzone; oleocanthal; olivetol; ortho-benzylphenol; orthocaine; ortho-cresol; ortho-phenylphenol; osajin; osalmid; oxybenzone; oxyphenbutazone; para-anol; para-benzylphenol; para-cresol; para-tert-pentyl-phenol; para-vinylguaiacol; paroxypropione; parvaquone; perezone; phenolphthalol; phenylacetylrinvanil; phloretin; piceatannol; pinoresinol; pinosylvin; pinosylvin monomethyl ether; pomiferin; probucol; propofol; propyl gallate; propylparaben; protocatechualdehyde; PSB-SB-487; pseudoisoeugenol; pterostilbene; raspberry ketone; resacetophenone; resveratrol; rottlerin; rottlerin 5,7-dimethyl ether; salicyl alcohol; salicylaldehyde; salicylanilide; sappanol; scillavone B; secoisolariciresinol; selligueain A; shogaol; silybin A; silybin B; silychristin; silydianin; sinapyl alcohol; sophoradin; sparassol; stilbestrol; strobilurin F; sulfuretin; syringaldehyde; syringaresinol; syringol; tetrahydrorottlerin; thunberginol F; thunberginol G; thymol; tithonine; tolcapone; tyrosol; ubiquinol; uliginosin A; vanillin; vanillyl alcohol; xanthohumol; xanthoxylin; xibornol; zingerone; 1,7-bis(4-hydroxyphenyl)-1,4,6-heptatrien-3-one; 11-hydroxyyangonin; 11-methoxy-12-hydroxydehydrokavain; 2,5-di-tert-pentyl-hydroquinone; 3-methoxy-4-hydroxyphenylglycol; 4,6-di-tert-butyl-meta-cresol; 4′-fluorocannabidiol; 4-hexylresorcinol; 4-hydroxyphenylacetaldehyde; 4-O-methylhonokiol; or 5-chloro-2-hydroxybenzophenone.

In some embodiments, the anion has a general structure Id or Ie.

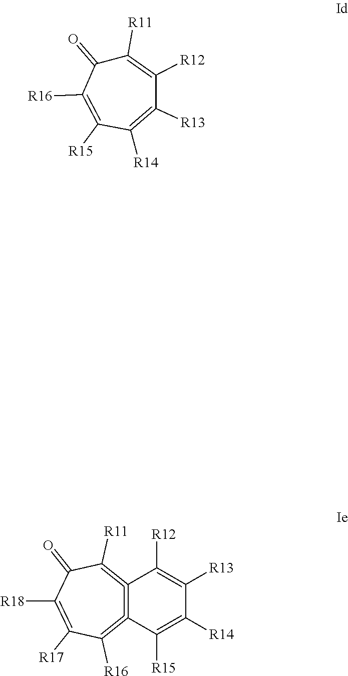

In some embodiments, general structures Id and Ie each comprise one oxide group, wherein one of R11, R12, R13, R14, R15, R16, R17, and R18 is oxide; one or two of R11, R12, R13, R14, R15, R16, R17, and R18 are selected hydro, methyl, ethyl, propyl, prop-2-yl, prop-1-enyl, prop-2-enyl, isoprenyl, and geranyl; and every other one of R11, R12, R13, R14, R15, R16, R17, and R18 is selected from hydro, hydroxy, and methoxy. In the case of general structure Id, which lacks R17 and R18, one of R11, R12, R13, R14, R15, and R16 is oxide; one or two of R11, R12, R13, R14, R15, and R16 are selected hydro, methyl, ethyl, propyl, prop-2-yl, prop-1-enyl, prop-2-enyl, isoprenyl, and geranyl; and every other one of R11, R12, R13, R14, R15, and R16 is selected from hydro, hydroxy, and methoxy. In some specific embodiments, the molecule is anthranol; procerin; purpurogallin; alpha-thujaplicin; beta-thujaplicin; or gamma-thujaplicin.

In some embodiments, the anion has a general structure IIa or IIb.

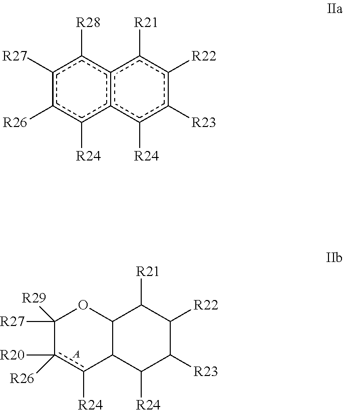

In some embodiments, (i) exactly one of R21, R22, R23, R24, R25, and R26 is oxide; (ii) exactly one of R21, R22, R23, R24, R25, R26, R27, and R28 is selected from hydro; methyl; ethyl; propyl; butyl; pentyl; hexyl; heptyl; isoprenyl; geranyl; 2-methylnonan-2-yl; 3,7,11,15-tetramethylhexadec-2-enyl; 1-hydroxy-4-methylpent-3-enyl; cyclohexyl; [4-(2-methylprop-2-yl)cyclohexyl]methyl; {5-oxo-1,3-dihydroxy-6,6-dimethyl-4-[(prop-2-yl)carbonyl]cyclohexa-1,3-dien-2-yl}methyl; 4-(4-chlorophenyl)cyclohexyl; phenyl; benzyl; 2-phenylethyl; 8-formyl-1,6,7-trihydroxy-5-(prop-2-yl)-3-methylnaphthalen-2-yl; 7-hydroxy-2H-chromene-3-yl; and 2,2-dimethyl-5-hydroxy-2H-chromen-8-yl, and every other one of R21, R22, R23, R24, R25, R26, R27, and R28 is selected from hydro; hydroxy; methyl; ethyl; propyl; prop-2-yl; methoxy; formyl; acetyl; 2-oxopropyl; (prop yl)carbonyl; and oxo; and (iii) R21, R22, R23, R24, R25, R26, R27, and R28 comprise exactly 0, 1, or 2 oxo groups. In the case of general structure IIb, which lacks R28, (i) exactly one of R21, R22, R23, R24, R25, and R26 is oxide; (ii) exactly one of R21, R22, R23, R24, R25, R26, and R27 is selected from hydro; methyl; ethyl; propyl; butyl; pentyl; hexyl; heptyl; isoprenyl; geranyl; 2-methylnonan-2-yl; 3,7,11,15-tetramethylhexadec-2-enyl; 1-hydroxy-4-methylpent-3-enyl; cyclohexyl; [4-(2-methylprop-2-yl)cyclohexyl]methyl; {5-oxo-1,3-dihydroxy-6,6-dimethyl-4-[(prop-2-yl)carbonyl]cyclohexa-1,3-dien-2-yl}methyl; 4-(4-chlorophenyl)cyclohexyl; phenyl; benzyl; 2-phenylethyl; 8-formyl-1,6,7-trihydroxy-5-(prop-2-yl)-3-methylnaphthalen-2-yl; 7-hydroxy-2H-chromene-3-yl; and 2,2-dimethyl-5-hydroxy-2H-chromen-8-yl, and every other one of R21, R22, R23, R24, R25, R26, and R27 is selected from hydro; hydroxy; methyl; ethyl; propyl; prop-2-yl; methoxy; formyl; acetyl; 2-oxopropyl; (prop-2-yl)carbonyl; and oxo; and (iii) R21, R22, R23, R24, R25, R26, and R27 comprise exactly 0, 1, or 2 oxo groups. General structure IIa comprises exactly 10 implicit carbon atoms that are each depicted by a junction of three lines in general structure IIa; and each dotted line in general structure IIa depicts an optional double bond that is selected such that each of the 10 implicit carbon atoms is bonded to exactly one other atom of general structure IIa with a double bond. General structure IIb comprises a dotted line that is labeled with the letter “A” that depicts (i) a required double bond when R26 is oxide; (ii) a required single bond when either R25 or R26 is oxo; and (iii) an optional double bond when R26 is neither oxo nor oxide. In some embodiments, when R26 is oxo or the dotted line that is labeled with the letter “A” depicts a double bond, then R20 is absent. In some embodiments, when R26 is not oxo and the dotted line that is labeled with the letter “A” depicts a single bond, then R20 is selected from hydro; 4-hydroxyphenyl; (2-hydroxyphenyl)methyl; and (3,4-dihydroxyphenyl)methyl. In some embodiments, when R27 is oxo, then R29 is absent; and when R27 is not oxo, then R29 is selected from hydro; methyl; 4-methylpent-3-enyl; 4,8,12-trimethyltridecyl; and 4,8,12-trimethyltrideca-3,7,11-trieneyl. In some specific embodiments, the molecule is alkannin; atovaquone; buparvaquone; cannabichromene; dehydroequol; glabrene; gossypol; javanicin; juglone; lapachol; lawsone; menadiol; naphthazarin; naphthoresorcinol; 2-naphthol; phthiocol; phylloquinol; plumbagin; PSB-SB-487; sappanol; spinochrome B; or uliginosin B. In some specific embodiments, the molecule is alpha-tocopherol; beta-tocopherol; gamma-tocopherol; delta-tocopherol; zeta2-tocopherol; eta-tocopherol; alpha-tocotrienol; beta-tocotrienol; gamma-tocotrienol; or delta-tocotrienol.

In some embodiments, the anion has a general structure IIIa or IIIb.

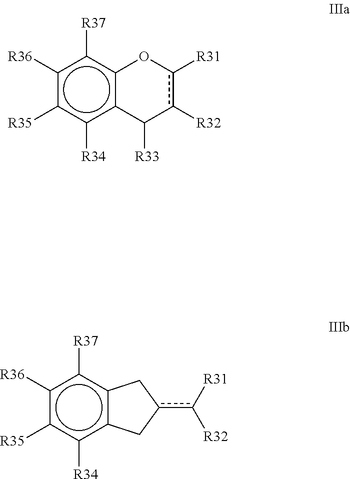

In some embodiments, one of R31 and R32 is selected from phenyl; 2-hydroxyphenyl; 3-hydroxyphenyl; 4-hydroxyphenyl; 2,4-dihydroxyphenyl; 2,6-dihydroxyphenyl; 3,4-dihydroxyphenyl; 3,5-dihydroxyphenyl; 3,4,5-trihydroxyphenyl; 3-hydroxy-4-methoxyphenyl; 4-hydroxy-3-methoxyphenyl; 3,4-dihydroxy-5-methyoxyphenyl; 3,5-dihydroxy-4-methyoxyphenyl; 3-hydroxy-4,5-dimethoxyphenyl; 4-hydroxy-3,5-dimethoxyphenyl; 2-methoxyphenyl; 3-methoxyphenyl; 4-methoxyphenyl; 2,4-dimethoxyphenyl; 2,6-dimethoxyphenyl; 3,4-dimethoxyphenyl; 3,5-dimethoxyphenyl; 3,4,5-trimethoxyphenyl; (3,4-dihydroxyphenyl)methyl; 1,3-benzodioxol-5-yl; 4,6-dimethoxy-3,5,11-trimethyltrideca-7,9,11-trienyl; 5-hydroxy-3,4-dimethoxybicyclo[4.2.0]octa-1,3,5-triene-1,7-diyl; and 2-oxo-3-hydroxy-10-(4-hydroxy-3-methoxyphenyl)-4-oxatricyclo[4.3.1.0^{3,7}]dec-8-en-8-yl, and the other one of R31 and R32 is selected from hydro; methyl; isoprenyl; geranyl; hydroxy; methoxy; and [(3,4,5-trihydroxyphenyl)carbonyl]oxy. In some embodiments, R33 is selected from hydro; hydroxy; and oxo. In some embodiments, one of R34, R35, R36, and R37 is an oxide group, and the other three of R34, R35, R36, and R37 are each independently selected from hydro; methyl; isoprenyl; geranyl; 5-methyl-2-(prop-1-en-2-yl)hex-4-enyl; 3,7-dimethylocta-2,6-dien-1-yl; hydroxy; and methoxy. Each dotted line in general structure IIIa and IIIb depicts an optional double bond. In some specific embodiments, the molecule is acacetin; acerosin; afzelechin; alnetin; ampelopsin; apiforol; apigenin; aromadendrin; artocarpetin; aureusidin; axillarin; azaleatin; baicalein; baptigenin; biochanin A; blumeatin; butin; calycosin; cannflavin A; cannflavin B; cannflavin C; catechin; chrysin; chrysoeriol; cirsilineol; cirsiliol; cirsimaritin; corymbosin; coumafuryl; daidzein; datiscetin; derrubone; dihydrokaempferide; dihydrokaempferol; dihydromorin; 4′,7-dihydroxyflavone; 7,8-dihydroxyflavone; diosmetin; echioidinin; epiafzelechin; epicatechin; epicatechin gallate; epigallocatechin; epigallocatechin gallate; equol; eriodictyol; ermanin; eupatilin; eupatorin; FBL-03G; fisetin; fisetinidol; formononetin; fustin; galangin; gallocatechin; garbanzol; gardenin A; gardenin B; gardenin C; gardenin D; gardenin E; genistein; 5 methylgenistein; genkwanin; geraldone; glycitein; gossypetin; guibourtinidol; hesperetin; hispidulin; homoeriodictyol; 6-hydroxyflavone; hymenoxin; hypolaetin; irigenin; isorhamnetin; isosakuranetin; isoscutellarein; isosilychristin; isoxanthohumol; jaceosidin; kaempferol; 4′ methylkaempferol; laricitrin; leucocyanidin; leucofisetinidin; leucopelargonidin; leucopeonidin; liquiritigenin; luteoforol; luteolin; 6-hydroxyluteolin; luteone; mearnsetin; meciadanol; melacacidin; mesquitol; methylchrysin; mikanin; morin; myricetin; naringenin; negletein; nepetin; nevadensin; nodifloretin; norartocarpetin; norwogonin; 0-806; onopordin; oritin; orobol; oroxylin A; ourateacatechin; pachypodol; pectolinarigenin; pedalitin; pilloin; pinobanksin; pinocembrin; pinostrobin; poriol; pratensein; pratol; primetin; primuletin; prunetin; pseudobaptigenin; psi-tectorigenin; quercetagetin; quercetin; retusin; rhamnazin; rhamnetin; robinetinidol; sakuranetin; scaposin; scillavone A; scillavone B; scutellarein; serpyllin; silychristin; silydianin; sophoraflavanone G; sorbifolin; spinacetin; sterubin; stigmatellin; sudachitin; sulfuretin; syringetin; taxifolin; techtochrysin; tectorigenin; tithonine; tricetin; tricin; velutin; wighteone; wightin; wogonin; xanthomicrol; or zapotinin.

In some embodiments, the anion has a general structure IV.

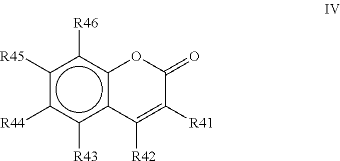

In some embodiments, (i) exactly one of R41, R42, R43, R44, R45, and R46 is an oxide group; (ii) exactly one of R41, R42, R43, R44, R45, and R46 is selected from hydro; isoprenyl; geranyl; 1-phenylpropyl; (3,7-dimethyloct-2-enyl)oxy; 3-oxo-1-(furan-2-yl)butyl; (2-oxo-4-hydroxy-2H-chromen-3-yl)methyl; 1-(2-oxo-4-hydroxy-2H-chromen-3-yl)ethyl; 2-oxo-2-ethoxy-1-(2-oxo-4-hydroxy-2H-chromen-3-yl)ethyl; 3-hydroxy-3-[4-(4-bromophenyl)phenyl]-1-phenylpropyl; 3-hydroxy-3-(4-chlorophenyl)-1-(5-chlorothiophene-2-yl)propyl; 1,2,3,4-tetrahydronaphthalen-1-yl; 3-(4-phenyl)phenyl-1,2,3,4-tetrahydronaphthalen-1-yl; 3-[4-(4-bromophenyl)phenyl]-1,2,3,4-tetrahydronaphthalen-1-yl; and 3-[4-({[4-(trifluoromethyl)phenyl]methyl}oxy)phenyl]-1,2,3,4-tetrahydronaphthalen-1-yl; and (iii) every other one of R41, R42, R43, R44, R45, and R46 is selected from hydro; hydroxy; methyl; and methoxy. In some specific embodiments, the molecule is brodifacoum; bromadiolone; coumatetralyl; daphnetin; dicoumarol; difenacoum; esculetin; ethyl biscoumacetate; ethylidene dicoumarol; ferujol; flocoumafen; fraxetin; 4-hydroxycoumarin; hymecromone; ostruthin; phenprocoumon; scopoletin; tioclomarol; or umbelliferone.

In some embodiments, the anion has a general structure Va or Vb.

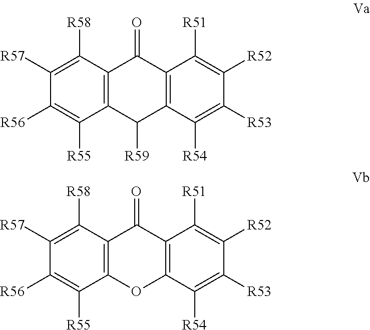

In some embodiments, (i) exactly one of R51, R52, R53, R54, R55, R56, R57, and R58 is an oxide group; exactly one or two of R51, R52, R53, R54, R55, R56, R57, and R58 is selected from hydro, isoprenyl, and geranyl; and every other one of R51, R52, R53, R54, R55, R56, R57, and R58 is selected from hydro, hydroxy, methyl, hydroxymethyl, methoxy, and formyl; and (ii) R59 is selected from hydro and oxo. In some specific embodiments, the molecule is alizarin; alizarin 1-methyl ether; alizarin 2-methyl ether; aloe emodin; anthragallol; anthralin; anthrapurpurin; 1,6-dihydroxyanthraquinone; anthrarobin; anthrarufin; beta-mangostin; chrysarobin; 9-hydroxychrysarobin; 3-hydroxychrysazin; damnacanthal; danthron; emodin; euxanthone; flavopurpurin; gamma-mangostin; 3,6-dimethylmangostin; 6-deoxy-gamma mangostin; gentisin; mangostin; oxyanthrarufin; oxychrysazin; parietin; purpurin; purpurin 1-methyl ether; purpurin 2,4-dimethyl ether; purpurin 2-methyl ether; purpuroxanthin; quinalizarin; quinizarin; or rubiadin.

In some embodiments, the anion has a general structure VI.

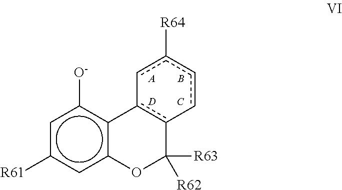

In some embodiments, R61 is selected from hydro; methyl; ethyl; propyl; butyl; pentyl; hexyl; heptyl; octyl; nonyl; decyl; prop-2-yl; but-2-yl; pent-2-yl; hex-2-yl; hept-2-yl; octan-2-yl; nonan yl; decan-2-yl; 2-methylpropyl; 2-methylbutyl; 2-methylpentyl; 2-methylhexyl; 2-methylheptyl; 2-methyloctyl; 2-methylnonyl; 2-methyldecyl; 2-methylprop-2-yl; 2-methylbut-2-yl; 2-methylpent yl; 2-methylhex-2-yl; 2-methylhept-2-yl; 2-methyloctan-2-yl; 2-methylnonan-2-yl; 2-methyldecan-2-yl; 3-methylbut-2-yl; 3-methylpent-2-yl; 3-methylhex-2-yl; 3-methylhept-2-yl; 3-methyloctan yl; 3-methylnonan-2-yl; 3-methyldecan-2-yl; 2,3-dimethylbut-2-yl; 2,3-dimethylpent-2-yl; 2,3-dimethylhex-2-yl; 2,3-dimethylhept-2-yl; 2,3-dimethyloctan-2-yl; 2,3-dimethylnonan-2-yl; 2,3-dimethyldecan-2-yl; cyclopropyl; 1-methylcyclopropyl; 1-ethylcyclopropyl; 1-propylcyclopropyl; 1-butylcyclopropyl; 1-pentylcyclopropyl; 1-hexylcyclopropyl; 1-heptylcyclopropyl; 1-octylcyclopropyl; 1-nonylcyclopropyl; cyclobutyl; 1-methylcyclobutyl; 1-ethylcyclobutyl; 1-propylcyclobutyl; 1-butylcyclobutyl; 1-pentylcyclobutyl; 1-hexylcyclobutyl; 1-heptylcyclobutyl; 1-octylcyclobutyl; cyclopentyl; 1-methylcyclopentyl; 1-ethylcyclopentyl; 1-propylcyclopentyl; 1-butylcyclopentyl; 1-pentylcyclopentyl; 1-hexylcyclopentyl; 1-heptylcyclopentyl; cyclohexyl; 1-methylcyclohexyl; 1-ethylcyclohexyl; 1-propylcyclohexyl; 1-butylcyclohexyl; 1-pentylcyclohexyl; 1-hexylcyclohexyl; ethenyl; prop-1-enyl; but-1-enyl; pent-1-enyl; hex-1-enyl; hept-1-enyl; octan-1-enyl; nonan-1-enyl; decan-1-enyl; ethynyl; prop-1-ynyl; but-1-ynyl; pent-1-ynyl; hex-1-ynyl; hept-1-ynyl; octan-1-ynyl; nonan-1-ynyl; decan-1-ynyl; 2-phenylethyl; 2-phenylprop-2-yl; adamant-1-yl; adamant-2-yl; 6-bromohex-2-enyl; 6-bromohex-2-ynyl; 2-methyl-6-bromohex-2-yl; 6-cyanohex-2-enyl; and 6-cyanohex-2-ynyl. In some embodiments, R62 is selected from hydro and methyl. In some embodiments, R63 is selected from hydro; methyl; 3-hydroxypropyl; 3-hydroxyprop-1-enyl; and 3-hydroxyprop-1-ynyl. In some embodiments, R64 is selected from hydro; methyl; hydroxy; hydroxymethyl; and oxo. In some embodiments, the dotted lines that are labeled with A, B, C, and D in general structure VI depict four optional double bonds that are selected such that A, B, C, and D depict either (i) zero double bonds; (ii) one double bond that occurs at the dotted line that is labeled with either A, B, or D; or (iii) three double bonds that occur at the dotted lines that are labeled with A, C, and D. In some specific embodiments, the molecule is AM-087; AM-2389; AM-4030; AM-411; AM-905; AM-906; AM-919; AM-938; AMG-1; AMG-36; AMG-41; canbisol; cannabinol; 11-nor-9beta-hydroxyhexahydrocannabinol; delta8-tetrahydrocannabinol; dexanabinol; dimethylheptylpyran; HU-210; HU-243; KM-233; nabilone; perrottetinene; synhexyl; tetrahydrocannabinol; 11-hydroxytetrahydrocannabinol; tetrahydrocannabinol-C4; tetrahydrocannabiorcol; tetrahydrocannabiphorol; or tetrahydrocannabivarin.

In some embodiments, the anion has a general structure VIIa or VIIb.

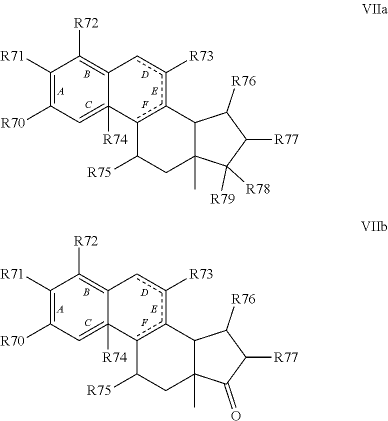

In some embodiments, R70 is selected from hydro, formyl, and cyano. In some embodiments, R71 is selected from oxo and oxide. When R70 is formyl or cyano, then R71 is oxide, R72 is hydro, and the dotted lines that are labelled with A, B, C, D, E, and F depict a single double bond that occurs at A. When R70 is hydro and R71 is oxo, then R72 is oxide, and the dotted lines that are labelled with A, B, C, D, E, and F depict a single double bond that occurs at B. When R70 is hydro and R71 is oxide, then R72 is selected from is hydro and hydroxy, and the dotted lines that are labelled with A, B, C, D, E, and F depict either (i) three double bonds that occur at A, B, and C, (ii) four double bonds that occur at either A, B, C, and E or A, B, C, and F, or (iii) five double bonds that occur at A, B, C, D, and F. In some embodiments, R73 is selected from hydro and 9-[(4,4,5,5,5-pentafluoropentyl)sulfinyl]nonanyl. In some embodiments, R74 is selected from hydro and methyl. In some embodiments, R75 is selected from hydro, hydroxy, and methoxy. In some embodiments, R76 and R77 are each independently selected from hydro and hydroxy. In some embodiments, R78 is selected from hydro, methyl, ethyl, ethenyl, and ethynyl. In some embodiments, R79 is selected from hydroxy, acetyloxy; (1-oxopropyl)oxy; (1-oxobutyl)oxy; (1-oxopentyl)oxy, (1-oxohexyl)oxy; (1-oxoheptyl)oxy, (1-oxooctanyl)oxy, (1-oxononanyl)oxy, (1-oxodecanyl)oxy, (1-oxoundecanyl)oxy, [1-oxo-3-(cyclopentyl)propyl]oxy, and [(2-methylprop-2-yl)amino]carbonyl. In some specific embodiments, R71 is oxide; R70 and R72 are hydro; and the dotted lines labeled with A, B, and C depict exactly 3 double bonds. In some very specific embodiments, the molecule is 17alpha-dihydroequilin; 17beta-dihydroequilin; 16-epiestriol; 17-epiestriol; 16,17-epiestriol; equilenin; equilin; estradiol; alpha-estradiol; estriol; estrone; ethinyl estradiol; formestane; fulvestrant; isoestradiol; 8-isoestrone; moxestrol; oxabolone; oxymesterone; or oxymetholone. In some very specific embodiments, the molecule is estradiol enanthate; estradiol undecylate; estradiol valerate; estradiol 17beta-cyclopentanepropanoate; or oxabolone 17-cyclopentanepropionate.

In some embodiments, the anion has a general structure VIIIa, VIIIb, VIIIc, VIIId, VIIIe, or VIIIf.

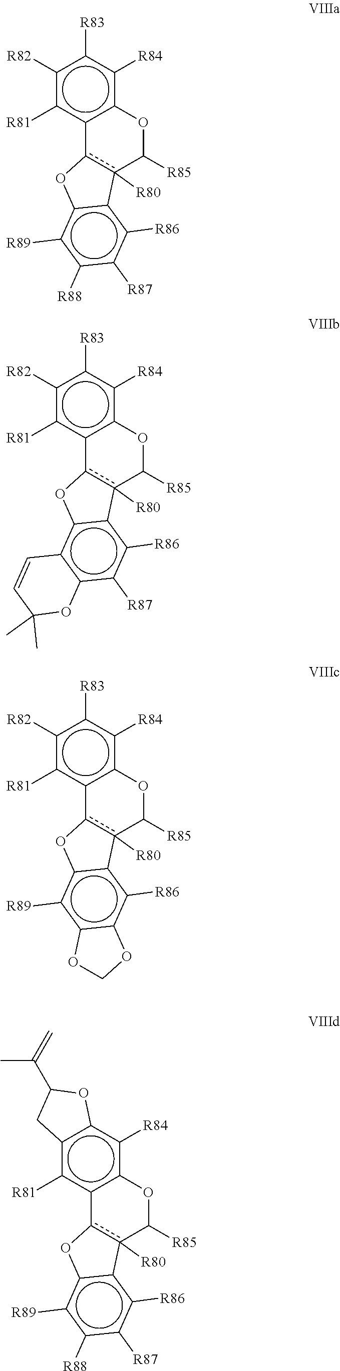

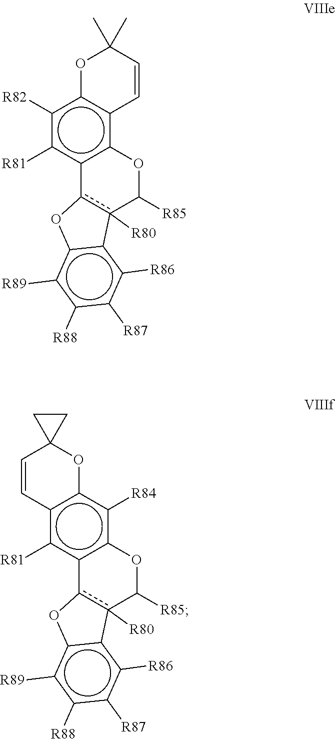

Each dotted line in general structures VIIIa, VIIIb, VIIIc, VIIId, VIIIe, and VIIIf depicts an optional double bond, wherein when the double bond is selected, then R80 is absent; and when the double bond is not selected, then R80 is selected from hydro and hydroxy. General structures VIIIa, VIIIb, VIIIc, VIIId, VIIIe, and VIIIf each comprise one oxide group, wherein one of R81, R82, R83, R84, R86, R87, R88, and R89 is oxide; one or two of R81, R82, R83, R84, R86, R87, R88, and R89 are selected from hydro, isoprenyl, geranyl, and 4-hydroxy-3-methylbutyl; and every other one of R81, R82, R83, R84, R86, R87, R88, and R89 is selected from hydro, hydroxy, and methoxy. In some embodiments, when R80 is absent or hydro, then R85 is selected from hydro, hydroxy, and oxo; and when R80 is hydroxy, then R85 is hydro. In some specific embodiments, the double bond in general structures VIIIa, VIIIb, VIIIc, VIIId, VIIIe, and VIIIf is selected, and R85 is selected from hydro and oxo. In some specific embodiments, double bond in general structures VIIIa, VIIIb, VIIIc, VIIId, VIIIe, and VIIIf is not selected, and R85 is hydro. In some specific embodiments, either R83 is oxide and R88 is hydroxy or methoxy; R88 is oxide and R83 is hydroxy or methoxy; or R83 and R88 are each independently selected from hydroxy and methoxy. In some specific embodiments, R86 is hydro. In some very specific embodiments, the molecule is cabenegrin A-I, cabenegrin A-II, coumestrol, glyceollin I, glyceollin II, glyceollin III, glyceollin IV, glycinol, glycyrrhizol A, medicagol, medicarpin, phaseolin, plicadin, psoralidin, or wedelolactone.

In some embodiments, the anion has a general structure IXa, IXb, IXc, IXd, IXe, IXf, or IXg.

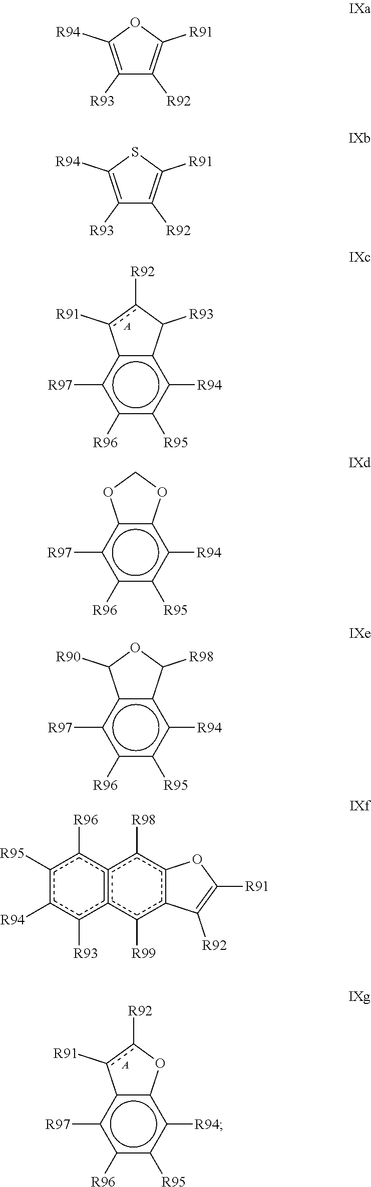

General structures IXa, IXb, IXc, IXd, IXe, IXf, and IXg each comprise an oxide group, wherein (i) one of R91, R92, R93, R94, R9S, R96, and R97 is oxide; (ii) two of R91, R92, R93, R94, R95, R96, and R97 are independently selected from hydro, ethyl, propyl, prop-2-yl, ethenyl, prop-1-enyl, prop-2-enyl, propen-2-yl, 2-methylprop-1-enyl, isoprenyl, formyl, acetyl, (ethoxy)carbonyl, (propoxy)carbonyl, 2-hydroxprop-2-yl, phenyl, 4-hydroxyphenyl, 3,4-dihydroxyphenyl, 3-hydroxy-4-methoxyphenyl, 4-hydroxy-3-methoxyphenyl, 4-oxo-3,5,7-trihydroxy-2,3-dihydro-4H-chromen-2-yl, 2-[1-oxo-7,8-dihydroxy-3-(methoxycarbonyl)-1H-isochromen-6-yl]ethyl, fluoro, chloro, bromo, and iodo; and (iii) every other one of R91, R92, R93, R94, R95, R96, and R97 is selected from hydro, hydroxy, hydroxymethyl, methoxy, and methyl. In some embodiments, R90 is selected from hydro, hydroxy, oxo, (phenyl)methylidene, (4-hydroxyphenyl)methylidene, and (3,4-hydroxyphenyl)methylidene. In some embodiments, R98 is selected from hydro, hydroxy, and oxo; when R98 is oxo, then R99 is oxo; and when R98 is hydro or hydroxy, then R99 is independently selected from hydro and hydroxy. Each dotted line labeled with “A” in general structures IXc and IXg is an optional double bond. General structure IXf comprises exactly 12 implicit carbon atoms that are each depicted by a junction of three lines in general structure IXf; and each dotted line in general structure IXf depicts an optional double bond that is selected such that each of the 12 implicit carbon atoms is bonded to exactly one other atom of general structure IXf with a double bond. In some specific embodiments, the molecule is chlorindanol; collinomycin; euparin; isomaltol; isosilychristin; mutisianthol; protiofate; sesamol; silychristin; or thunberginol F.

In some embodiments, a composition disclosed anywhere in this patent document comprises water and hydroxide. In some embodiments, a composition disclosed anywhere in this patent document comprises ethanol and ethoxide. In some embodiments, a composition disclosed anywhere in this patent document comprises 1,2-propanediol and one or both of 1-hydroxypropane-2-oxide and 2-hydroxypropane-1-oxide. In some embodiments, a composition disclosed anywhere in this patent document comprises 1,2,3-propanetriol and one or both of 1,3-dihydroxypropane-2-oxide and 2,3-dihydroxypropane-1-oxide. In some embodiments, a composition disclosed anywhere in this patent document comprises lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); copper (II) cation (“Cu++”); ammonium (“NH₄+”); protonated ethanolamine; choline; protonated lysine; protonated arginine; or protonated sphingosine. In some specific embodiments, a composition disclosed anywhere in this patent document comprises sodium cation. In some specific embodiments, a composition comprises potassium cation.

In some embodiments, the subject is a rodent, lagomorph, feline, canine, porcine, ovine, caprine, lama, vicugna, bovine, equine, or primate. In some very specific embodiments, the subject is human.

What is claimed is:

1. A method to sedate or anesthetize a subject, comprising providing a composition comprising 2,6-diisopropylphenolate and administering the composition to the subject in an amount that is effective to sedate or anesthetize the subject.

2. The method of claim 1, wherein the composition comprises water; the 2,6-diisopropylphenolate is dissolved in the water; and the water has a pH that is greater than 8.5 when the composition is provided.

3. The method of claim 1 or 2, wherein the composition comprises the 2,6-diisopropylphenolate at a concentration of at least 15 grams per liter.

4. The method of any one of claims 1-3, wherein the composition lacks triglycerides, fatty acids, and phospholipids at a combined concentration greater than 50 grams per liter.

5. The method of any one of claims 1-4, wherein the composition comprises 2,6-diisopropylphenol, and the composition comprises the 2,6-diisopropylphenolate at a greater molar concentration than the 2,6-diisopropylphenol when the composition is provided.

6. The method of any one of claims 1-5, comprising contacting the 2,6-diisopropylphenolate with a Brønsted acid to convert the 2,6-diisopropylphenolate into 2,6-diisopropylphenol.

7. The method of any one of claims 1-6, comprising contacting the composition with a liquid comprising bicarbonate to convert the 2,6-diisopropylphenolate into 2,6-diisopropylphenol.

8. The method of any one of claims 1-7, comprising converting the 2,6-diisopropylphenolate into 2,6-diisopropylphenol in situ subsequent to administering the composition to the subject.

9. The method of any one of claims 1-8, wherein the amount comprises at least 0.5 micrograms and no greater than 5 milligrams of 2,6-diisopropylphenolate per kilogram bodyweight of the subject per minute.

10. The method of any one of claims 1-7, comprising converting the 2,6-diisopropylphenolate into 2,6-diisopropylphenol ex vivo prior to administering the composition to the subject.

11. The method of claim 10, wherein the amount comprises at least 0.5 micrograms and no greater than 5 milligrams of 2,6-diisopropylphenol per kilogram bodyweight of the subject per minute.

12. A method to modulate international normalized ratio (“INR”) in a subject, comprising providing a composition comprising 2-oxo-3-(1-phenylpropyl)-2H-chromen-4-oxide and administering the composition to the subject in an amount that is effective to either increase INR to greater than 1.2 in the subject or maintain INR at greater than 1.2 in the subject.

13. The method of claim 12, wherein the amount is effective to increase INR to at least 1.5 and no greater than 4.0 in the subject or maintain INR at at least 1.5 and no greater than 4.0 in the subject.

14. The method of claim 12 or 13, wherein the amount is effective to either prophylactically prevent or treat (a) thromboembolic events, (b) venous thrombus, (c) pulmonary embolism, (d) thromboembolic complications associated with atrial fibrillation or cardiac valve replacement in the subject, (e) myocardial infarction, (f) ischemic stroke, (g) ischemia-related death, or (h) two or more of the foregoing in the subject.

15. The method of any one of claims 12-14, wherein the composition comprises 2-oxo-3-(1-phenylpropyl)-2H-chromen-4-ol, and the composition comprises the 2-oxo-3-(1-phenylpropyl)-2H-chromen-4-oxide at a greater molar concentration than the 2-oxo-3-(1-phenylpropyl)-2H-chromen-4-ol when the composition is provided.

16. The method of any one of claims 12-15, comprising contacting the 2-oxo-3-(1-phenylpropyl)-2H-chromen-4-oxide with a Brønsted acid to convert the 2-oxo-3-(1-phenylpropyl)-2H-chromen-4-oxide into 2-oxo-3-(1-phenylpropyl)-2H-chromen-4-ol.

17. The method of any one of claims 12-16, comprising converting the 2-oxo-3-(1-phenylpropyl)-2H-chromen-4-oxide into 2-oxo-3-(1-phenylpropyl)-2H-chromen-4-ol in situ subsequent to administering the composition to the subject.

18. The method of any one of claims 12-17, wherein the amount comprises at least 0.1 milligrams and no greater than 20 milligrams of 2-oxo-3-(1-phenylpropyl)-2H-chromen-4-oxide per day.

19. The method of any one of claims 12-16, comprising converting the 2-oxo-3-(1-phenylpropyl)-2H-chromen-4-oxide into 2-oxo-3-(1-phenylpropyl)-2H-chromen-4-ol ex vivo prior to administering the composition to the subject.

20. The method of claim 19, wherein the amount comprises at least 0.1 milligrams and no greater than 20 milligrams of 2-oxo-3-(1-phenylpropyl)-2H-chromen-4-ol per day.

21. The method of any one of claims 12-20, wherein the composition is formulated for oral administration; the composition is formulated to allow the conversion of the 2-oxo-3-(1-phenylpropyl)-2H-chromen-4-oxide into 2-oxo-3-(1-phenylpropyl)-2H-chromen-4-ol before the 2-oxo-3-(1-phenylpropyl)-2H-chromen-4-oxide reaches the stomach of the subject; and the composition is formulated to allow absorption of the 2-oxo-3-(1-phenylpropyl)-2H-chromen-4-ol by the epithelial lining of the gastrointestinal tract between the lips and the stomach, excluding the stomach and the outer surfaces of the lips, and including the esophagus and the inner surfaces of the lips.

22. The method of any one of claims 12-20, wherein the administering is inhalational administering or injecting.

23. The method of any one of claims 12-22, wherein the composition is a liquid, and the 2-oxo (1-phenylpropyl)-2H-chromen-4-oxide is dissolved in the liquid.

24. A method to increase estrogen in a subject, comprising providing a composition comprising an estrogen anion and administering the composition to the subject in an amount that is effective to increase estrogen in the subject, wherein the estrogen anion is (8R,9S,13S,14S,17S)-17-hydroxy-13-methyl-6,7,8,9,11,12,14,15,16,17-decahydrocyclopenta[a]phenanthrene-3-oxide; (8R,9S,13S,14S,17R)-17-ethynyl-17-hydroxy-13-methyl-7,8,9,11,12,14,15,16-octahydro-6H-cyclopenta[a]phenanthrene-3-oxide; or (8R,9S,13S,14S,16R,17R)-16,17-dihydroxy-13-methyl-6,7,8,9,11,12,14,15,16,17-decahydrocyclopenta[a]phenanthrene-3-oxide.

25. The method of claim 24, comprising contacting the estrogen anion with a Brønsted acid to convert the estrogen anion into an estrogen molecule.

26. A method to administer an estrogen to a subject, comprising providing a composition comprising an estrogen anion and orally administering the composition to the subject, wherein: the estrogen anion has a conjugate acid that is an estrogen molecule; the estrogen molecule has an acid dissociation constant in water of at least 50 femtomolar and no greater than 50 nanomolar for conversion of the estrogen molecule into the estrogen anion; the composition is formulated to allow the conversion of the estrogen anion into the estrogen molecule before the estrogen anion reaches the stomach of the subject; and the composition is formulated to allow absorption of the estrogen molecule by the epithelial lining of the gastrointestinal tract between the lips and the stomach, excluding the stomach and the outer surfaces of the lips, and including the esophagus and the inner surfaces of the lips.

27. The method of claim 26, wherein:
the estrogen anion is (8R,9S,13S,14S,17S)-13-methyl-17-[(1-oxopentyl)oxy]-6,7,8,9,11,12,14,15,16,17-decahydrocyclopenta[a]phenanthrene-3-oxide, and the estrogen molecule is (8R,9S,13S,14S,17S)-13-methyl-17-[(1-oxopentyl)oxy]-6,7,8,9,11,12,14,15,16,17-decahydrocyclopenta[a]phenanthren-3-ol;
the estrogen anion is (8R,9S,13S,14S,17S)-13-methyl-17-[(1-oxo-3-cyclopentylpropyl)oxy]-6,7,8,9,11,12,14,15,16,17-decahydrocyclopenta[a]phenanthrene-3-oxide, and the estrogen molecule is (8R,9S,13S,14S,17S)-13-methyl-17-[(1-oxo-3-cyclopentylpropyl)oxy]-6,7,8,9,11,12,14,15,16,17-decahydrocyclopenta[a]phenanthren-3-ol;
the estrogen anion is (8R,9S,13S,14S,17S)-13-methyl-17-[(1-oxoheptyl)oxy]-6,7,8,9,11,12,14,15,16,17-decahydrocyclopenta[a]phenanthrene-3-oxide, and the estrogen molecule is (8R,9S,13S,14S,17S)-13-methyl-17-[(1-oxoheptyl)oxy]-6,7,8,9,11,12,14,15,16,17-decahydrocyclopenta[a]phenanthren-3-ol;
the estrogen anion is (8R,9S,13S,14S,17S)-13-methyl-17-[(1-oxoundecanyl)oxy]-6,7,8,9,11,12,14,15,16,17-decahydrocyclopenta[a]phenanthrene-3-oxide, and the estrogen molecule is (8R,9S,13S,14S,17S)-13-methyl-17-[(1-oxoundecanyl)oxy]-6,7,8,9,11,12,14,15,16,17-decahydrocyclopenta[a]phenanthren-3-ol;
the estrogen anion is (8R,9S,13S,14S,17R)-17-hydroxy-13-methyl-6,7,8,9,11,12,14,15,16,17-decahydrocyclopenta[a]phenanthrene-3-oxide, and the estrogen molecule is (8R,9S,13S,14S,17R)-13-methyl-6,7,8,9,11,12,14,15,16,17-decahydrocyclopenta[a]phenanthrene-3,17-diol;
the estrogen anion is (8S,9S,11S,13S,14S,17R)-17-ethynyl-17-hydroxy-11-methoxy-13-methyl-7,8,9,11,12,14,15,16-octahydro-6H-cyclopenta[a]phenanthrene-3-oxide, and the estrogen molecule is (8S,9S,11S,13S,14S,17R)-17-ethynyl-11-methoxy-13-methyl-7,8,9,11,12,14,15,16-octahydro-6H-cyclopenta[a]phenanthren-3,17-diol;
the estrogen anion is (8R,9S,13S,14S,16S,17R)-16,17-dihydroxy-13-methyl-6,7,8,9,11,12,14,15,16,17-decahydrocyclopenta[a]phenanthrene-3-oxide, and the estrogen molecule is (8R,9S,13S,14S,16S,17R)-13-methyl-6,7,8,9,11,12,14,15,16,17-decahydrocyclopenta[a]phenanthrene-3,16,17-triol;
the estrogen anion is (8R,9S,13S,14S,16S,17S)-16,17-dihydroxy-13-methyl-6,7,8,9,11,12,14,15,16,17-decahydrocyclopenta[a]phenanthrene-3-oxide, and the estrogen molecule is (8R,9S,13S,14S,16S,17S)-13-methyl-6,7,8,9,11,12,14,15,16,17-decahydrocyclopenta[a]phenanthrene-3,16,17-triol;
the estrogen anion is (8R,9S,13S,14S)-17-oxo-13-methyl-7,8,9,11,12,14,15,16-octahydro-6H-cyclopenta[a]phenanthrene-3-oxide, and the estrogen molecule is (8R,9S,13S,14S)-17-oxo-13-methyl-7,8,9,11,12,14,15,16-octahydro-6H-cyclopenta[a]phenanthrene-3-ol;
the estrogen anion is (9S,13S,14S)-17-oxo-13-methyl-9,11,12,14,15,16-hexahydro-6H-cyclopenta[a]phenanthrene-3-oxide, and the estrogen molecule is (9S,13S,14S)-17-oxo-13-methyl-9,11,12,14,15,16-hexahydro-6H-cyclopenta[a]phenanthrene-3-ol;
the estrogen anion is (13 S,14S)-17-oxo-13-methyl-7,11,12,14,15,16-hexahydro-6H-cyclopenta[a]phenanthrene-3-oxide, and the estrogen molecule is (13S,14S)-17-oxo-13-methyl-7,11,12,14,15,16-hexahydro-6H-cyclopenta[a]phenanthrene-3-ol;
the estrogen anion is (13 S,14S)-17-oxo-13-methyl-12,14,15,16-tetrahydro-11H-cyclopenta[a]phenanthrene-3-oxide, and the estrogen molecule is (13S,14S)-17-oxo-13-methyl-12,14,15,16-tetrahydro-11H-cyclopenta[a]phenanthrene-3-ol;
the estrogen anion is (9S,13S,14S,17S)-17-hydroxy-13-methyl-6,9,11,12,14,15,16,17-octahydrocyclopenta[a]phenanthrene-3-oxide, and the estrogen molecule is (9S,13S,14S,17S)-13-methyl-6,9,11,12,14,15,16,17-octahydrocyclopenta[a]phenanthrene-3,17-diol;
the estrogen anion is (9S,13S,14S,17R)-17-hydroxy-13-methyl-6,9,11,12,14,15,16,17-octahydrocyclopenta[a]phenanthrene-3-oxide, and the estrogen molecule is (9S,13S,14S,17R)-13-methyl-6,9,11,12,14,15,16,17-octahydrocyclopenta[a]phenanthrene-3,17-diol;
the estrogen anion is (13S,14S,17S)-17-hydroxy-13-methyl-6,7,11,12,14,15,16,17-octahydrocyclopenta[a]phenanthrene-3-oxide, and the estrogen molecule is (13S,14S,17S)-13-methyl-6,7,11,12,14,15,16,17-octahydrocyclopenta[a]phenanthrene-3,17-diol;
the estrogen anion is (13S,14S,17R)-17-hydroxy-13-methyl-6,7,11,12,14,15,16,17-octahydrocyclopenta[a]phenanthrene-3-oxide, and the estrogen molecule is (13S,14S,17R)-13-methyl-6,7,11,12,14,15,16,17-octahydrocyclopenta[a]phenanthrene-3,17-diol;
the estrogen anion is (13S,14S,17S)-17-hydroxy-13-methyl-11,12,14,15,16,17-hexahydrocyclopenta[a]phenanthrene-3-oxide, and the estrogen molecule is (13S,14S,17S)-13-methyl-11,12,14,15,16,17-hexahydrocyclopenta[a]phenanthrene-3,17-diol;
the estrogen anion is (13S,14S,17R)-17-hydroxy-13-methyl-11,12,14,15,16,17-hexahydrocyclopenta[a]phenanthrene-3-oxide, and the estrogen molecule is (13S,14S,17R)-13-methyl-11,12,14,15,16,17-hexahydrocyclopenta[a]phenanthrene-3,17-diol;
the estrogen anion is (8R,9S,13S,14S,15R,16R,17R)-15,16,17-trihydroxy-13-methyl-6,7,8,9,11,12,14,15,16,17-decahydrocyclopenta[a]phenanthrene-3-oxide, and the estrogen molecule is (8R,9S,13S,14S,15R,16R,17R)-13-methyl-6,7,8,9,11,12,14,15,16,17-decahydrocyclopenta[a]phenanthrene-3,15,16,17-tetrol;
the estrogen anion is 4-[4-(4-hydroxyphenyl)hexa-2,4-dien-3-yl]phenolate, and the estrogen molecule is 4-[4-(4-hydroxyphenyl)hexa-2,4-dien-3-yl]phenol;
the estrogen anion is 4-[4-(4-hydroxyphenyl)hex-3-en-3-yl]phenolate, and the estrogen molecule is 4-[4-(4-hydroxyphenyl)hex-3-en-3-yl]phenol;
the estrogen anion is 4-[4-(4-methoxyphenyl)hex-3-en-3-yl]phenolate, and the estrogen molecule is 4-[4-(4-methoxyphenyl)hex-3-en-3-yl]phenol;
the estrogen anion is 4-(4-{4-[(phenylmethyl)oxy]phenyl}hex-3-en-3-yl)phenolate, and the estrogen molecule is 4-(4-{4-[(phenylmethyl)oxy]phenyl}hex-3-en-3-yl)phenol;
the estrogen anion is 4-[2-(4-hydroxyphenyl)vinyl]phenolate, and the estrogen molecule is 4-[2-(4-hydroxyphenyl)vinyl]phenol;
the estrogen anion is 4-[3-(4-hydroxyphenyl)but-2-en-2-yl]phenolate, and the estrogen molecule is 4-[3-(4-hydroxyphenyl)but-2-en-2-yl]phenol;
the estrogen anion is 4-[4-(4-hydroxyphenyl)hex-3-yl]phenolate, and the estrogen molecule is 4-[4-(4-hydroxyphenyl)hex-3-yl]phenol;
the estrogen anion is 4-[3-ethyl-4-(4-hydroxyphenyl)hex-2-yl]phenolate, and the estrogen molecule is 4-[3-ethyl-4-(4-hydroxyphenyl)hex-2-yl]phenol;
the estrogen anion is 4-[4-ethyl-5-(4-hydroxyphenyl)hex-3-yl]phenolate, and the estrogen molecule is 4-[4-ethyl-5-(4-hydroxyphenyl)hex-3-yl]phenol; or
the estrogen anion is 4-[4-(4-hydroxy-3-methylphenyl)hex-3-yl]-2-methylphenolate; and the estrogen molecule is 4-[4-(4-hydroxy-3-methylphenyl)hex-3-yl]-2-methylphenol.

28. The method of claim 25 or 26, wherein:
the estrogen anion is (8R,9S,13S,14S,17S)-17-hydroxy-13-methyl-6,7,8,9,11,12,14,15,16,17-decahydrocyclopenta[a]phenanthrene-3-oxide, and the estrogen molecule is (8R,9S,13S,14S,17S)-13-methyl-6,7,8,9,11,12,14,15,16,17-decahydrocyclopenta[a]phenanthrene-3,17-diol;
the estrogen anion is (8R,9S,13S,14S,17R)-17-ethynyl-17-hydroxy-13-methyl-7,8,9,11,12,14,15,16-octahydro-6H-cyclopenta[a]phenanthrene-3-oxide, and the estrogen molecule is (8R,9S,13S,14S,17R)-17-ethynyl-13-methyl-7,8,9,11,12,14,15,16-octahydro-6H-cyclopenta[a]phenanthrene-3,17-diol; or
the estrogen anion is (8R,9S,13S,14S,16R,17R)-16,17-dihydroxy-13-methyl-6,7,8,9,11,12,14,15,16,17-decahydrocyclopenta[a]phenanthrene-3-oxide, and the estrogen molecule is (8R,9S,13S,14S,16R,17R)-13-methyl-6,7,8,9,11,12,14,15,16,17-decahydrocyclopenta[a]phenanthrene-3,16,17-triol.

29. The method of any one of claims 25-28, wherein the composition comprises the estrogen molecule; and the composition comprises the estrogen anion at a greater molar concentration than the estrogen molecule when the composition is provided.

30. The method of any one of claims 25-29, comprising converting the estrogen anion into the estrogen molecule in situ subsequent to administering the composition to the subject.

31. The method of any one of claims 25-29, comprising converting the estrogen anion into the estrogen molecule ex vivo prior to administering the composition to the subject.

32. The method of any one of claims 24-31, wherein the amount is effective either as a contraceptive, to treat symptoms of menstruation, to treat dysmenorrhea, to treat menorrhagia, to treat polycystic ovary syndrome, to treat endometriosis, to treat female hypogonadism, to treat absence of menstruation, to treat symptoms of menopause, to provide perimenopausal or postmenopausal hormone replacement therapy, to provide feminizing hormone therapy, to treat hirsutism, to treat acne, or two or more of the foregoing.

33. The method of any one of claims 24-32, wherein the amount is effective to treat a psychiatric condition or a neurodegenerative disease.

34. The method of any one of claims 24-33, wherein the amount is effective to treat anxiety, schizophreniform disorder, schizophrenia, multiple sclerosis, mild cognitive impairment, or Alzheimer's disease.

35. The method of any one of claims 24-34, wherein the amount is effective to treat inflammation, an autoimmune disease, or sepsis.

36. The method of any one of claims 24-35, wherein the amount is effective to treat arthritis, ankylosing spondylitis, an inflammatory autoimmune-mediated arthritis, rheumatoid arthritis, psoriatic arthritis, psoriasis, plaque psoriasis, lupus, Sjogren's syndrome, inflammatory bowel disease, Crohn's disease, or ulcerative colitis.

37. The method of any one of claims 24-36, wherein the amount is effective to treat breast cancer, prostate cancer, or benign prostatic hyperplasia.

38. The method of any one of claims 24-37, wherein the composition is a liquid, and the estrogen anion is dissolved in the liquid.

39. A method to administer a cannabinoid to a subject, comprising providing a composition comprising a cannabinoid anion and administering the composition to the subject.

40. The method of claim 39, wherein the cannabinoid anion is 3-hydroxy-2-[(1R,6R)-6-isopropenyl-3-methylcyclohex-2-enyl]-5-pentylphenolate; 3-hydroxy-2-[(1R,6R)-6-isopropenyl-3-methylcyclohex-2-enyl]-5-propylphenolate; 2-geranyl-3-hydroxy-5-pentylphenolate; 2-geranyl-3-hydroxy-5-propylphenolate; (6aR,10aR)-6,6,9-trimethyl-3-pentyl-6a,7,8,10a-tetrahydro-6H-benzo[c]chromene-1-oxide; (6aR,10aR)-6,6,9-trimethyl-3-propyl-6a,7,8,10a-tetrahydro-6H-benzo[c]chromene-1-oxide; 6,6,9-trimethyl-3-pentyl-6H-benzo[c]chromene-1-oxide; or 6,6,9-trimethyl-3-propyl-6H-benzo[c]chromene-1-oxide.

41. The method of claim 39 or 40, comprising contacting the cannabinoid anion with a Brønsted acid to convert the cannabinoid anion into a cannabinoid molecule, wherein the cannabinoid molecule has an acid dissociation constant in water of at least 50 femtomolar and no greater than 50 nanomolar for conversion of the cannabinoid molecule into the cannabinoid anion.

42. The method of any one of claims 39-41, wherein the administering is either oral or topical administering.

43. A method to administer a cannabinoid to a subject, comprising providing a composition comprising a cannabinoid anion and orally administering the composition to the subject, wherein: the cannabinoid anion has a conjugate acid that is a cannabinoid molecule; the cannabinoid molecule has an acid dissociation constant in water of at least 50 femtomolar and no greater than 50 nanomolar for conversion of the cannabinoid molecule into the cannabinoid anion; the composition is formulated to allow the conversion of the cannabinoid anion into the cannabinoid molecule before the cannabinoid anion reaches the stomach of the subject; and the composition is formulated to allow absorption of the cannabinoid molecule by the epithelial lining of the gastrointestinal tract between the lips and the stomach, excluding the stomach and the outer surfaces of the lips, and including the esophagus and the inner surfaces of the lips.

44. The method of any one of claims 41-43, wherein the composition comprises the cannabinoid molecule; and the composition comprises the cannabinoid anion at a greater molar concentration than the cannabinoid molecule when the composition is provided.

45. The method of any one of claims 41-44, comprising converting the cannabinoid anion into the cannabinoid molecule in situ subsequent to administering the composition to the subject.

46. The method of any one of claims 41-44, comprising converting the cannabinoid anion into the cannabinoid molecule ex vivo prior to administering the composition to the subject.

47. The method of any one of claims 41-46, wherein:
the cannabinoid anion is 3-hydroxy-2-[(1R,6R)-6-isopropenyl-3-methylcyclohex-2-enyl]-5-pentylphenolate, and the cannabinoid molecule is 2-[(1R,6R)-6-isopropenyl-3-methylcyclohex-2-enyl]-5-pentylbenzene-1,3-diol;
the cannabinoid anion is 3-hydroxy-2-[(1R,6R)-6-isopropenyl-3-methylcyclohex-2-enyl]-5-propylphenolate, and the cannabinoid molecule is 2-[(1R,6R)-6-isopropenyl-3-methylcyclohex-2-enyl]-5-propylbenzene-1,3-diol;
the cannabinoid anion is 2-geranyl-3-hydroxy-5-pentylphenolate, and the cannabinoid molecule is 2-geranyl-5-pentylbenzene-1,3-diol;
the cannabinoid anion is 2-geranyl-3-hydroxy-5-propylphenolate, and the cannabinoid molecule is 2-geranyl-5-propylbenzene-1,3-diol;
the cannabinoid anion is (6aR,10aR)-6,6,9-trimethyl-3-pentyl-6a,7,8,10a-tetrahydro-6H-benzo[c]chromene-1-oxide, and the cannabinoid molecule is (6aR,10aR)-6,6,9-trimethyl-3-pentyl-6a,7,8,10a-tetrahydro-6H-benzo[c]chromene-1-ol;
the cannabinoid anion is (6aR,10aR)-6,6,9-trimethyl-3-propyl-6a,7,8,10a-tetrahydro-6H-benzo[c]chromene-1-oxide, and the cannabinoid molecule is (6aR,10aR)-6,6,9-trimethyl-3-propyl-6a,7,8,10a-tetrahydro-6H-benzo[c]chromene-1-ol;
the cannabinoid anion is 6,6,9-trimethyl-3-pentyl-6H-benzo[c]chromene-1-oxide, and the cannabinoid molecule is 6,6,9-trimethyl-3-pentyl-6H-benzo[c]chromene-1-ol; or
the cannabinoid anion is 6,6,9-trimethyl-3-propyl-6H-benzo[c]chromene-1-oxide, and the cannabinoid molecule is 6,6,9-trimethyl-3-propyl-6H-benzo[c]chromene-1-ol.

48. The method of any one of claims 39-47, wherein the cannabinoid anion is 3-hydroxy-2-[(1R,6R)-6-isopropenyl-3-methylcyclohex-2-enyl]-5-pentylphenolate or 3-hydroxy-2-[(1R,6R)-6-isopropenyl-3-methylcyclohex-2-enyl]-5-propylphenolate.

49. The method of any one of claims 39-48, wherein the composition is a liquid, and the cannabinoid anion is dissolved in the liquid.

50. The method of any one of claims 39-49, wherein the amount is effective to prophylactically prevent or treat muscle cramping, muscle spasms, restless-legs syndrome, nystagmus, a dyskinetic movement disorder, tremor, seizures, epilepsy, muscular dystrophy, or inclusion body myositis.

51. The method of any one of claims 39-50, comprising administering the composition to the subject while the subject is having an active seizure, wherein the composition is effective to either arrest or reduce the severity of the active seizure.

52. The method of any one of claims 39-51, wherein the amount is effective to reduce blood pressure in the subject.

53. The method of any one of claims 39-52, wherein the amount is effective to prophylactically prevent or treat prehypertension or hypertension.

54. The method of any one of claims 39-53, wherein the amount is effective to treat attention deficit hyperactivity disorder (“ADHD”), autism or an autism spectrum disorder, Asperger syndrome, fragile X syndrome, Down syndrome, a pervasive developmental disorder not otherwise specified (“PDD-NOS”), a childhood disintegrative disorder, or Tourette's syndrome.

55. The method of any one of claims 39-54, wherein the amount is effective to treat anxiety, post-traumatic stress disorder (“PTSD”), depression, bipolar disorder, obsessive-compulsive disorder, schizophreniform disorder, schizophrenia, or psychosis.

56. The method of any one of claims 39-55, wherein the amount is effective to treat pain or inflammation.

57. The method of any one of claims 39-56, wherein the amount is effective to treat an autoimmune disorder.

58. The method of any one of claims 39-57, wherein the amount is effective to treat arthritis, ankylosing spondylitis, an inflammatory autoimmune-mediated arthritis, rheumatoid arthritis, psoriatic arthritis, psoriasis, plaque psoriasis, lupus, Sjogren's syndrome, inflammatory bowel disease, Crohn's disease, or ulcerative colitis.

59. The method of any one of claims 39-58 wherein the amount is effective to treat a neurodegenerative disease.

60. The method of any one of claims 39-59, wherein the amount is effective to treat Parkinson's Disease, and treating the Parkinson's Disease comprises treating Parkinsonian tremor.

61. The method of any one of claims 39-60, wherein the amount is effective to treat multiple sclerosis, mild cognitive impairment, Alzheimer's Disease, amyotrophic lateral sclerosis (“ALS”), or Huntington's disease.

62. The method of any one of claims 39-61, wherein the amount is effective to treat obesity, metabolic syndrome, or diabetes mellitus.

63. The method of any one of claims 39-62, wherein the amount is effective to treat a viral infection or a bacterial infection.

64. The method of any one of claims 39-63, wherein the amount is effective to treat an infection caused by Escherichia coli, Klebsiella pneumoniae, Pseudomonas aeruginosa, Acinetobacter baumannii, Moraxella catarrhalis, Legionella pneumophila, Staphylococcus aureus, Streptococcus pneumoniae, Enterococcus faecium, Clostridioides difficile, Mycobacterium tuberculosis, Neisseria gonorrhoeae, Cutibacterium acnes, or COVID-19.

65. A method to prophylactically prevent or treat seizures in a subject, comprising providing a composition comprising 3-hydroxy-2-[(1R,6R)-6-isopropenyl-3-methylcyclohex-2-enyl]-5-pentylphenolate or 3-hydroxy-2-[(1R,6R)-6-isopropenyl-3-methylcyclohex-2-enyl]-5-propylphenolate, and administering the composition to the subject in an amount that is effective to prophylactically prevent or treat seizures.

66. A method to administer a substituted phenol to a subject, comprising providing a composition comprising a substituted phenolate and orally administering the composition to the subject, wherein: the substituted phenolate has a conjugate acid that is the substituted phenol; the substituted phenol has an acid dissociation constant in water of at least 50 femtomolar and no greater than 50 nanomolar for conversion of the substituted phenol into the substituted phenolate; the composition is formulated to allow the conversion of the substituted phenolate into the substituted phenol before the substituted phenolate reaches the stomach of the subject; and the composition is formulated to allow absorption of the substituted phenol by the epithelial lining of the gastrointestinal tract between the lips and the stomach, excluding the stomach and the outer surfaces of the lips, and including the esophagus and the inner surfaces of the lips.

67. The method of claim 66, comprising converting the substituted phenolate into the substituted phenol in situ subsequent to administering the composition to the subject.

68. The method of claim 66, comprising converting the substituted phenolate into the substituted phenol ex vivo prior to administering the composition to the subject.

69. The method of any one of claims 66-68, wherein the composition comprises the substituted phenol, and the composition comprises the substituted phenolate at a greater molar concentration than the substituted phenol when the composition is provided.

70. The method of any one of claims 66-69, wherein the substituted phenol is 2-methoxy-4-(prop-2-enyl)phenol, and the substituted phenolate is 2-methoxy-4-(prop-2-enyl)phenolate.

71. The method of any one of claims 66-69, wherein the substituted phenol is 5-methyl-2-(prop-2-yl)phenol, and the substituted phenolate is 5-methyl-2-(prop-2-yl)phenolate.

72. The method of any one of claims 66-69, wherein the substituted phenol is 2-methyl-5-(prop-2-yl)phenol, and the substituted phenolate is 2-methyl-5-(prop-2-yl)phenolate.

73. The method of any one of claims 66-69, wherein the substituted phenol is 2-methoxy-4-({N-[(6E)-1-oxo-8-methylnon-6-enyl]amino}methyl)phenol, and the substituted phenolate is 2-methoxy-4-({N-[(6E)-1-oxo-8-methylnon-6-enyl]amino}methyl)phenolate.

74. The method of any one of claims 66-69, wherein the substituted phenol is 2-methoxy-4-[(5S)-3-oxo-5-hydroxydecanyl]phenol, and the substituted phenolate is 2-methoxy-4-[(5S)-3-oxo-5-hydroxydecanyl]phenolate.

75. The method of any one of claims 66-69, wherein the substituted phenol is 4-formyl-2-methoxyphenol, and the substituted phenolate is 4-formyl-2-methoxyphenolate.

76. The method of any one of claims 66-69, wherein the substituted phenol is 4-formyl-2-ethoxyphenol, and the substituted phenolate is 4-formyl-2-ethoxyphenolate.

77. The method of any one of claims 66-69, wherein the substituted phenol is 4-(3-oxobutyl)phenol, and the substituted phenolate is 4-(3-oxobutyl)phenolate.

78. The method of any one of claims 66-69, wherein the substituted phenol is 2-methoxy-4-[3,5-dioxo-7-(4-hydroxy-3-methoxyphenyl)hepta-1,6-dieneyl]phenol, and the substituted phenolate is 2-methoxy-4-[3,5-dioxo-7-(4-hydroxy-3-methoxyphenyl)hepta-1,6-dieneyl]phenolate.

79. The method of any one of claims 66-69, wherein the substituted phenol is 2,6-diisopropylphenol, and the substituted phenolate is 2,6-diisopropylphenolate.

80. A method to administer a molecule to a subject, comprising providing a composition comprising an anion; converting the anion into the molecule; and administering the composition to the subject, wherein the molecule has an acid dissociation constant in water of at least 50 femtomolar and no greater than 50 nanomolar for conversion of the molecule into the anion.

81. The method of claim 80, wherein the anion is dissolved in the composition at a concentration that is greater than the solubility of the molecule in water.

82. The method of claim 80 or 81, wherein the molecule has an octanol-water partition coefficient that is greater than 1.

83. The method of any one of claims 80-82, wherein administering is orally administering; the composition is formulated to allow the conversion of the anion into the molecule before the anion reaches the stomach of the subject; and the composition is formulated to allow absorption of the molecule by the epithelial lining of the gastrointestinal tract between the lips and the stomach, excluding the stomach and the outer surfaces of the lips, and including the esophagus and the inner surfaces of the lips.

84. The method of any one of claims 80-83, comprising converting the anion into the molecule in situ subsequent to administering the composition to the subject.

85. The method of any one of claims 80-83, comprising converting the anion into the molecule ex vivo prior to administering the composition to the subject.

86. The method of any one of claims 80-85, wherein the composition comprises the molecule, and the composition comprises the anion at a greater molar concentration than the molecule when the composition is provided.

87. The method of any one of claims 80-86, wherein the anion has a general structure Ia, Ib, or Ic 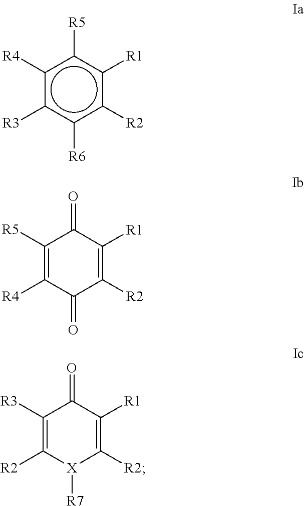
one of R1, R2, R3, and R4 is oxide;
one of R1, R2, R3, R4, R5 and R6 is selected from hydro; hydroxy; methoxy; fluoro; chloro; bromo; and iodo;
one of R1, R2, R3, R4, R5 and R6 is selected from hydro; hydroxy; hydroxymethyl; 2-hydroxyethyl; 1,2-dihydroxyethyl; 3-hydroxyprop-1-enyl; methyl; 2-methylprop-2-yl; methoxy; ethoxy; propoxy; butoxy; pentoxy; hexoxy; heptoxy; octanoxy; (prop-2-yl)oxy; isoprenyloxy; benzyloxy; [4-(prop-2-enyl)phenyl]oxy; fluoro; chloro; bromo; iodo; amino; and nitro;
one of R1, R2, R3, R4, R5 and R6 is selected from hydro; hydroxy; methoxy; formyl; acetyl; 2-oxoethyl; 1-oxopropyl; 1-oxobutyl; (prop-2-yl)carbonyl; 3-oxobutyl; 3-oxobut-1-enyl; (methoxy)carbonyl; (ethoxy)carbonyl; (propoxy)carbonyl; (2-propoxy)carbonyl; (butoxy)carbonyl; (pentoxy)carbonyl; (hexoxy)carbonyl; and (heptoxy)carbonyl;
one of R1, R2, R3, R4, R5 and R6 is selected from hydro; methoxy; methyl; ethyl; propyl; butyl; pentyl; hexyl; heptyl; octanyl; nonanyl; decanyl; undecanyl; dodecanyl; tridecyl; tetradecyl; pentadecyl; prop-2-yl; but-2-yl; pent-2-yl; hex-2-yl; hept-2-yl; octan-2-yl; nonan-2-yl; decan-2-yl; 2-methylprop-2-yl; 2-methylbut-2-yl; 2-methylpent-2-yl; 2-methylhex-2-yl; 2-methylhept-2-yl; 2-methyloctan-2-yl; 2-methylnonan-2-yl; 2-methyldecan-2-yl; 3-methylbut-2-yl; 3-methylpent-2-yl; 3-methylhex-2-yl; 3-methylhept-2-yl; 3-methyloctan-2-yl; 3-methylnonan-2-yl; 3-methyldecan-2-yl; 2,4,4-trimethylpent-2-yl; vinyl; prop-1-enyl; prop-2-enyl; pentadec-8-enyl; 2-methylbut-1-en-3-yl; 3-methylbut-1-en-3-yl; 3-ethenyl-3,7-dimethylocta-1,6-dienyl; 4-ethenyl-4,7-dimethylocta-1,6-dien-2-yl; isoprenyl; geranyl; 3,7,11,15,19,23,27,31,35,39-decamethyltetraconta-2,6,10,14,18,22,26,30,34,38-decaenyl; phenyl; benzyl; and 2-phenylethyl;
one of R1, R2, R3, R4, R5 and R6 is selected from hydro; methyl; ethyl; propyl; 2-methylprop-2-yl; 2-methylbut-2-yl; isoprenyl; geranyl; 6-methylhept-5-en-2-yl; 6-methoxy-5-[(methoxy)carbonyl]-4-methylhexa-1,3,5-trienyl; 2-{[1-oxo-4-formyl-3-(2-oxoethyl)hex-4-enyl]oxy}ethyl; 3-oxobutyl; 3-oxobut-1-enyl; 3-oxooct-4-enyl; 3-oxodec-4-enyl; 3-oxododec-4-enyl; 3-oxotetradec-4-enyl; 3-oxohexadec-4-enyl; 3-oxo-5-hydroxyoctanyl; 3-oxo-5-hydroxydecanyl; 3-oxo-5-hydroxydodecanyl; 3-oxo-5-hydroxytetradecanyl; 3-oxo-5-hydroxyhexadecanyl; [N-(1-oxononanyl)amino]methyl; {N-[1-oxo-12-(1-oxo-2-phenylethyl)octadec-9-enyl]amino}methyl; [N-(1-oxo-8-methylnon-6-enyl)amino]methyl; [N-(1-oxo-8-methylnonanyl)amino]methyl; [N-(1-oxo-7-methyloctanyl)amino]methyl; [N-(1-oxo methyldec-6-enyl)amino]methyl; [N-(1-oxo-9-methyldecanyl)amino]methyl; [N-(1-oxo methyldec-6-enyl)amino]methyl; 2-{[4-(4-hydroxyphenyl)but-2-yl]amino}ethyl; 3-{[2-(3,4-dihydroxyphenyl)ethyl]amino}butyl; 1-hydroxy-2-[1-(4-hydroxyphenyl)prop-2-yl]amino ethyl; 2-[2-hydroxy-2-(3,5-dihydroxyphenyl)ethyl]amino propyl; 4-1 [1-oxo-3,7-dimethyl-9-(2,6,6-trimethylcyclohex-1-enyl)non-2,4,6,8-tetraenyl]amino; cyclohexyl; cycloheptyl; adamant-1-yl; adamant-2-yl; 3,3-dimethylcycolhexyl; 6,6-dimethyl-4-oxo-2-bicyclo[3.1.1]heptanyl; 3-hydroxycyclohexyl; 5-hydroxy-2-(3-hydroxypropyl)cyclohexyl; 6-(prop-1-en-2-yl)-3-methylcyclohex-2-enyl; 6-(prop-2-yl)-3-methylcyclohex-2-enyl; 6-(prop-1-en-2-yl)-3-methylcyclohex-3-enyl; 6-(prop-2-yl)-3-methylcyclohex-3-enyl; 4-hydroxy-3-(prop-2-enyl)phenyl; 6-hydroxy-3-(prop-2-enyl)phenyl; 4-methoxy-3-(prop-2-enyl)phenyl; (4-hydroxyphenyl)methyl; 2-(4-hydroxyphenyl)ethyl; 2-(3,5-dihydroxyphenyl)ethyl; 2-hydroxy-2-(3,4,5-trimethoxyphenyl)ethyl; 2-phenylethenyl; 2-(4-hydroxyphenyl)ethenyl; 2-(3,4-dihydroxyphenyl)ethenyl; 2-(3,5-dihydroxyphenyl)ethenyl; 2-(3,4,5-trimethoxyphenyl)ethenyl; 2-(4-hydroxyphenyl)prop-2-yl; 1-oxo-3-phenylpropyl; 1-oxo-3-(4-hydroxyphenyl)propyl; 3-oxo-3-(2,4,6-tri hydroxyphenyl)propyl; 1-oxo-3-phenylprop-2-enyl; 3-oxo-3-(4-hydroxyphenyl)prop-1-enyl; 1-oxo-3-(4-hydroxyphenyl)prop-2-enyl; 3-oxo-3-(2,4-dihydroxyphenyl)prop-1-enyl; 1-oxo-3-(3,4-dihydroxy-2-methoxyphenyl)prop-2-enyl; 1-oxo-3-[4-hydroxy-3,5-bis(isoprenyl)phenyl]prop-2-enyl; 3-oxo-3-[3,5-dihydroxy-4-isoprenylphenyl]prop-1-enyl; 3-oxo-3-[4,6-dihydroxy-2-methoxy-5-isoprenylphenyl]prop-1-enyl; 2-(4-hydroxyphenyl)but-2-yl; 2,3-dimethyl-4-(3,4-dihydroxyphenyl)butyl; 2,3-dimethyl-4-(4-hydroxy-3-methoxyphenyl)butyl; 2,3-bis(hydroxymethyl)-4-(3-hydroxyphenyl)butyl; 2,3-bis(hydroxymethyl)-4-(4-hydroxy-3-methoxyphenyl)butyl; 3-(4-hydroxyphenyl)but-2-en-2-yl; 4-(4-hydroxyphenyl)hex-3-yl; 4-(4-hydroxy-3-methylphenyl)hex-3-yl; 3-ethyl-4-(4-hydroxyphenyl)hex-2-yl; 4-ethyl-5-(4-hydroxyphenyl)hex-3-yl; 4-(4-hydroxyphenyl)hex-3-en-3-yl; 3-(4-hydroxyphenyl)-2-methylpent-1-enyl; 1-(4-hydroxyphenyl)-2-methylpent-1-en-3-yl; 4-(4-methoxyphenyl)hex-3-en-3-yl; 4-{4-[(phenylmethyl)oxy]phenyl}hex-3-en-3-yl; 4-(4-hydroxyphenyl)hexa-2,4-dien-3-yl; 3-oxo-7-(4-hydroxyphenyl)hepta-1,4,6-trieneyl; 3-oxo-7-(4-hydroxyphenyl)hepta-1,3,6-trieneyl; 7-(4-hydroxyphenyl)-3,5-dioxohepta-1,6-dieneyl; 7-(4-hydroxy-3-methoxyphenyl)-3,5-dioxohepta-1,6-dieneyl; [6-oxo-2,4-dihydroxy-3,3-dimethyl-5-(1-oxo-2-methylpropyl)cyclohexa-1,4-dienyl]methyl; [2,6-dihydroxy-4-methoxy-5-methyl-3-(1-oxo-2-methylpropyl)phenyl]methyl; [6-hydroxy-2,4-dimethoxy-5-methyl-3-(1-oxo-2-methylpropyl)phenyl]methyl; 1-oxo-3-[4-hydroxy methoxy-3-isoprenylphenyl]prop-2-enyl; 1-oxo-3-[4-hydroxy-2-methoxy-3-(2-methylbut-1-en yl)phenyl]prop-2-enyl; 1-oxo-3-[4-hydroxy-2-methoxy-5-(2-methylbut-1-en-3-yl)phenyl]prop enyl; 1-oxo-3-[4-hydroxy-2-methoxy-5-(3-methylbut-1-en-3-yl)phenyl]prop-2-enyl; 1-oxo-3-[4-hydroxy-3, 5-bis(isoprenyl)phenyl]prop-2-enyl; phenylcarbonyl; (4-methylphenyl)carbonyl; (2-hydroxyphenyl)carbonyl; (2,4-dihydroxyphenyl)carbonyl; (2-hydroxy-4-methoxyphenyl)carbonyl; [(3,3,5-trimethylcyclohexyl)oxy]carbonyl; [(3-hydroxy-4-methoxycarbonyl-2,5-dimethylphenyl)oxy]carbonyl; [(3-formyl-2,4-dihydroxy-6-methylphenyl)carbonyl]oxy; (4-hydroxyphenyl)-[2-(hydroxymethyl)phenyl]methyl; 1,7,7-trimethyl-2-bicyclo[2.2.1]heptyl; {4-oxo-5-[(3-hydroxyphenyl)methyl]-3-oxacyclopentyl}methyl; {2-oxo-5-[(3-hydroxyphenyl)methyl]-3-oxacyclopentyl}methyl; {2-oxo-3-[(3-methyoxy-4-hydroxyphenyl)methylidene]cyclohexylidene)methyl; 2-(4-methoxy-2-oxo-2H-pyran-6-yl)ethenyl; 4-[(4-hydroxy-3-methoxyphenyl)methyl]-3-(hydroxymethyl)tetrahydrofuran-2-yl; [2-(4-hydroxy-3-methoxyphenyl)-3-(hydroxymethyl)tetrahydrofuran-4-yl]methyl; {2-oxo-4-[(3,4-dimethoxyphenyl)methyl]tetrahydrofuran-3-yl}methyl; {2-oxo-4-[(3-methoxy-4-hydroxyphenyl)methyl]tetrahydrofuran-3-yl}methyl; {2-oxo-3-[(3-methoxy-4-hydroxyphenyl)methyl]tetrahydrofuran-4-yl}methyl; {2-oxo-4-[(3-methoxy-4-hydroxyphenyl)hydroxymethyl]tetrahydrofuran-3-yl}methyl; {2-oxo-3-[(3-methoxy-4-hydroxyphenyl)methyl]tetrahydrofuran-4-yl}hydroxymethyl; 7-[5,5-dimethyl-4-oxo-tetrahydrofuran-2-yl]-3-methylocta-2,6-dienyl; (1,2,4a-trimethyl-5-methylidene-3,4,6,7, 8,8a-hexahydro-2H-naphthalen-1-yl)methyl; 3-hydroxy-7-hydroxymethyl-1,2,3,4,4a, 5,6,7,8,8a-decahydronaphthalen-1-yl; 6-(4-hydroxy-3-methoxyphenyl)-1,3,3a,4,6,6a-hexahydrofuro[3,4-c]furan-3-yl; 6-(4-hydroxy-3, 5-dimethoxyphenyl)-1,3,3a,4,6,6a-hexahydrofuro[3,4-c]furan-3-yl; 7-oxo-4-hydroxy-5-methyl-5,6-dihydro-4H-2-benzofuran-1-yl; 3-hydroxymethyl-7-methoxy-5-(3-oxoprop-1-enyl)-2,3-dihydro-1-benzofuran-2-yl; (2-ethyl-1-benzofuran-3-yl)carbonyl; (3-oxo-4-hydroxy-2-benzofuran-1-ylidene)methyl; (3-oxo-6-hydroxy-1-benzofuran-2-ylidene)methyl; (3-oxo-4,6-dihydroxy-1-benzofuran-2-ylidene)methyl; (3-oxo-6-hydroxy-4-methoxy-1-benzofuran-2-ylidene)methyl; 4-(1,3-benzodioxol-5-yl)-2,3-dimethylbutyl; 8-oxo-4-hydroxy-5a,6,8a,9-tetrahydro-5H-[2]benzofuro[5,6-f][ 1,3]benzodioxol-9-yl; 7-hydroxy-2H-chromen-3-yl; 4-oxo-7-methoxy-4H-chromen-2-yl; 1-oxo-8-hydroxy-3,4-dihydro-1H-isochromen-3-yl; (3,4,7-trihydroxy-3,4-dihydro-2H-chromen-3-yl)methyl; (4-oxo-5-hydroxy-6,7-dimethoxy-2,3-dihydro-4H-chromen-3-yl)methyl; [2-oxo-5-hydroxy-7-(2-methylnonan-2-yl)-2H-chromen-3-yl]methyl; 5,7-dihydroxy-8-(1-oxo-3-phenylprop-2-enyl)-2,2-dimethyl-2H-chromen-6-yl]methyl; 5,7-dimethoxy-8-(1-oxo phenylprop-2-enyl)-2,2-dimethyl-2H-chromen-6-yl]methyl; 5,7-dihydroxy-8-(1-oxo-3-phenylprop-2-enyl)-2,2-dimethyl-3,4-dihydro-2H-chromen-6-yl; 4-oxo-5,7-dihydroxy-8-[2-hydroxy-5-(4-oxo-5,7-dihydroxy-4H-chromen-2-yl)phenyl]-4H-chromen-2-yl; 4-oxo-8-[6-(2,4-dihydroxybenzoyl) (2,4-dihydroxyphenyl)-3-methylcyclohex-2-enyl]-5,7-dihydroxy-3-isoprenyl-4H-chromen-2-yl; 8-oxo-2-hydroxymethyl-5-methoxy-2,3-dihydropyrano[2,3-h][1,4]benzodioxin-3-yl; 9-oxo methoxy-2-{[(acetyl)oxy]methyl}-2,3-dihydropyrano[3,2-h][1,4]benzodioxin-3-yl; 2-hydroxymethyl-7-(4-oxo-3,5,7-trihydroxy-2,3-dihydro-4H-chromen-2-yl)-2,3-dihydro-1,4-benzodioxin-3-yl; 8-oxo-9-hydroxy-[1,3]diolxolo[4,5-g]-4H-chromen-7-yl; 8-oxo-9-methoxy-[1,3]diolxolo[4,5-g]-4H-chromen-7-yl; 8,8-dimethyl-3,4-dihydro-2H-pyrano[2,3-f]chromen-3-yl; 4-oxo-5-hydroxy-6-isoprenyl-8,8-dimethylpyrano[2,3-h]-4H-chromen-3-yl; 7-hydroxy-3-hydroxymethyl-4-(4-oxo-3,5,7-trihydroxy-2,3-dihydro-4H-chromen-2-yl)-2,3-dihydro-1-benzofuran-2-yl; 7-hydroxy-3-hydroxymethyl-5-(4-oxo-3,5,7-trihydroxy-2,3-dihydro-4H-chromen-2-yl)-2,3-dihydro-1-benzofuran-2-yl; 2-oxo-3-hydroxy-8-(3,5,7-trihydroxy-4-oxo-2,3-dihydro-4H-chromen-2-yl)-4-oxatricyclo[4.3.1.0^{3,7}]dec-8-en-10-yl; 6,9,17,19,21-pentahydroxy-5-(4-hydroxyphenyl)-4,12,14-trioxapentacyclo[11.7.1.0^{2,11}.0^{3,8}.0^{15,20}]henicosa-2(11),3(8),9,15,17,19-hexaen-13-yl; 6,9,17,19,21-pentahydroxy-3-(4-hydroxyphenyl)-4,12,14-trioxapentacyclo[11.7.1.0^{2,11}. 0^{3,8}.0^{15,20}]henicosa-2(11),3(8),9,15,17,19-hexaen-5-yl; N-(4-phenyl)carbamoyl; N-(4-hydroxyphenyl)carbamoyl; [(2-hydroxyphenyl)carbonyl]amino; 1-oxo-icosa-5,8,11,14-tetraenyl)amino; N-(4-chlorophenyl)carbamoyl; 1-oxo-2-(2,6-dioxopiperidin-4-yl)ethyl; 1-hydroxy-2-{[4-(4-hydroxyphenyl)butyl]amino}ethyl; 4-{[2-hydroxy-2-(3,4-dihydroxyphenyl)ethyl]amino}butyl; [(4-oxo-1,4-dihydroquinolin-3-yl)carbonyl]amino; 3,5-dioxo-4-butyl-2-phenylpyrazolidin-1-yl; (2-{[4-hydroxy-3,5-di(2-methylprop-2-yl)phenyl]sulfanyl}propan-2-yl)sulfanyl; (3-chloro-6-hydroxyphenyl)methyl; (2,4-dichlorophenyl)methyl; 3-(3-fluoro-4-hydroxyphenyl)pent-2-yl; and 2-(3-fluoro-4-hydroxyphenyl)pent-3-yl;
X is selected from O and N, wherein when X is O, then R7 is absent; and when X is N, then R7 is selected from hydro and methyl; and
the anion has a molecular weight that is greater than 108 grams per mole.

88. The method of claim 87, wherein the anion has a general structure Ia; R1 is oxide; R2 is hydro, methyl, 2-methylprop-2-yl, geranyl, hydroxy, methoxy, ethoxy, hydroxymethyl, formyl, or amino; and each of R3, R5, and R6 is hydro.

89. The method of claim 87 or 88, wherein the anion has a general structure Ia; R1 is oxide; and at least 4 of R2, R3, R4, R5, and R6 are selected from hydro, hydroxy, and methoxy.

90. The method of any one of claims 80-87, wherein the molecule is abnormal cannabidiol; acetosyringone; actiphenol; adipostatin A; aleuritin; alpha-kosin; alpha-peltatin; AM404; amentoflavone; amylmetacresol; apocynin; arbutamine; arctigenin; ascofuranone; aspidinol; atranorin; aureusidin; bakuchiol; balanophonin; benzarone; benzbromarone; benzestrol; benziodarone; benzophenone-2; benzophenone-6; benzoresorcinol; beta-kosin; beta-resorcylaldehyde; bifluranol; bilobol; bisdemethoxycurcumin; bisphenol A; bisphenol B; bisphenol F; bromosalicylchloranilide; bromosaligenin; butylated hydroxyanisole; butylated hydroxytoluene; butylparaben; cannabicyclohexanol; cannabidiol; cannabidiphorol; cannabidivarin; cannabigerol; cannabigerovarin; canolol; capsaicin; carvacrol; chavibetol; chavicol; clofoctol; clorophene; combretastatin; combretastatin A-1; combretastatin A-4; combretastatin B-1; coniferyl alcohol; cotoin; CP 55,244; CP 55,940; (C6)-CP 47,497; (C7)-CP 47,497; (C9)-CP 47,497; curcumin; cyclovalone; DB-2073; deferiprone; dehydroequol; demethoxycurcumin; dianol; dichlorophen; dienestrol; diethylstilbestrol; diethylstilbestrol monobenzyl ether; dihydrocapsaicin; dihydrokanakugiol; dihydroresveratrol; dimethylheptyl cannabidiol; dimethylstilbestrol; dioxybenzone; dobutamine; DOPAL; DOPEG; drupanol; durantin A; embelin; enterodiol; enterolactone; ethyl maltol; ethyl vanillin; ethylparaben; eugenol; fenoterol; fenretinide; flopropione; fumigatin; gentisyl alcohol; geranin A; geranylhydroquinone; [6]-gingerol; glabridin; guaiacol; heminordihydroguaiaretic acid; heptylparaben; hexestrol; homocapsaicin I; homocapsaicin II; homodihydrocapsaicin; homosalate; homovanillyl alcohol; honokiol; HU-331; hydroxymatairesinol; hydroxytyrosol; ilimaquinone; irilone; irisolone; isoeugenol; isoliquiritigenin; isosilybin A; isosilybin B; isosilychristin; ivacaftor; kanakugiol; kuwanon G; lariciresinol; leptosidin; leptosphaerin A; leptosphaerin B; licochalcone A; licochalcone B; licochalcone C; licochalcone D; licochalcone E; licochalcone F; macelignan; magnolol; maltol; matairesinol; mequinol; mestilbol; meta-cresol; methestrol; methylparaben; mexenone; monobenzone; nonivamide; nordihydrocapsaicin; nordihydroguaiaretic acid; 0-1602; 0-1871; obovatol; octabenzone; oleocanthal; olivetol; ortho-benzylphenol; orthocaine; ortho-cresol; ortho-phenylphenol; osajin; osalmid; oxybenzone; oxyphenbutazone; para-anol; para-benzylphenol; para-cresol; para-tert-pentyl-phenol; para-vinylguaiacol; paroxypropione; parvaquone; perezone; phenolphthalol; phenylacetylrinvanil; phloretin; piceatannol; pinoresinol; pinosylvin; pinosylvin monomethyl ether; pomiferin; probucol; propofol; propyl gallate; propylparaben; protocatechualdehyde; PSB-SB-487; pseudoisoeugenol; pterostilbene; raspberry ketone; resacetophenone; resveratrol; rottlerin; rottlerin 5,7-dimethyl ether; salicyl alcohol; salicylaldehyde; salicylanilide; sappanol; scillavone B; secoisolariciresinol; selligueain A; shogaol; silybin A; silybin B; silychristin; silydianin; sinapyl alcohol; sophoradin; sparassol; stilbestrol; strobilurin F; sulfuretin; syringaldehyde; syringaresinol; syringol; tetrahydrorottlerin; thunberginol F; thunberginol G; thymol; tithonine; tolcapone; tyrosol; ubiquinol; uliginosin A; vanillin; vanillyl alcohol; xanthohumol; xanthoxylin; xibornol; zingerone; 1,7-bis(4-hydroxyphenyl)-1,4,6-heptatrien-3-one; 11-hydroxyyangonin; 11-methoxy-12-hydroxydehydrokavain; 2,5-di-tert-pentyl-hydroquinone; 3-methoxy-4-hydroxyphenylglycol; 4,6-di-tert-butyl-meta-cresol; 4′-fluorocannabidiol; 4-hexylresorcinol; 4-hydroxyphenylacetaldehyde; 4-O-methylhonokiol; or 5-chloro-2-hydroxybenzophenone.

91. The method of any one of claims 80-86, wherein the anion has a general structure Id or Ie 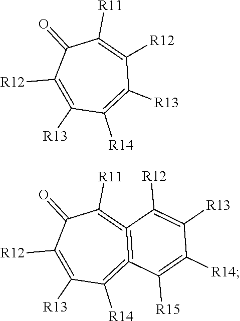
general structures Id and Ie each comprise one oxide group, wherein one of R11, R12, R13, R14, R15, R16, R17, and R18 is oxide;
one or two of R11, R12, R13, R14, R15, R16, R17, and R18 are selected hydro, methyl, ethyl, propyl, prop-2-yl, prop-1-enyl, prop-2-enyl, isoprenyl, and geranyl; and
every other one of R11, R12, R13, R14, R15, R16, R17, and R18 is selected from hydro, hydroxy, and methoxy.

92. The method of any one of claims 80-86 and 91, wherein the molecule is anthranol; procerin;
purpurogallin; alpha-thujaplicin; beta-thujaplicin; or gamma-thujaplicin.

93. The method of any one of claims 80-86, wherein the anion has a general structure IIa or Ilb 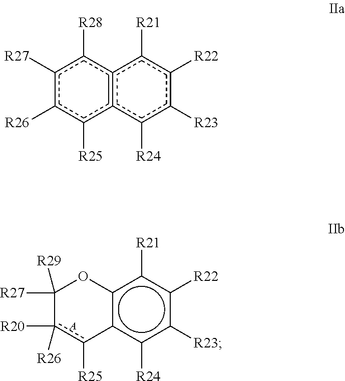
exactly one of R21, R22, R23, R24, R25, and R26 is oxide;
exactly one of R21, R22, R23, R24, R25, R26, R27, and R28 is selected from hydro; methyl; ethyl; propyl; butyl; pentyl; hexyl; heptyl; isoprenyl; geranyl; 2-methylnonan-2-yl; 3,7,11,15-tetramethylhexadec-2-enyl; 1-hydroxy-4-methylpent-3-enyl; cyclohexyl; [4-(2-methylprop yl)cyclohexyl]methyl; {5-oxo-1,3-dihydroxy-6,6-dimethyl-4-[(prop-2-yl)carbonyl]cyclohexa-1,3-dien-2-yl}methyl; 4-(4-chlorophenyl)cyclohexyl; phenyl; benzyl; 2-phenylethyl; 8-formyl-1,6,7-trihydroxy-5-(prop-2-yl)-3-methylnaphthalen-2-yl; 7-hydroxy-2H-chromene-3-yl; and 2,2-dimethyl-5-hydroxy-2H-chromen-8-yl, and every other one of R21, R22, R23, R24, R25, R26, R27, and R28 is selected from hydro; hydroxy; methyl; ethyl; propyl; prop-2-yl; methoxy; formyl; acetyl; 2-oxopropyl; (prop-2-yl)carbonyl; and oxo;
R21, R22, R23, R24, R25, R26, R27, and R28 comprise exactly 0, 1, or 2 oxo groups;
general structure IIa comprises exactly 10 implicit carbon atoms that are each depicted by a junction of three lines in general structure IIa;
each dotted line in general structure IIa depicts an optional double bond that is selected such that each of the 10 implicit carbon atoms is bonded to exactly one other atom of general structure IIa with a double bond;
general structure IIb comprises a dotted line that is labeled with the letter “A” and that depicts (i) a required double bond when R26 is oxide; (ii) a required single bond when either R25 or R26 is oxo; and (iii) an optional double bond when R26 is neither oxo nor oxide;
when R26 is oxo or the dotted line that is labeled with the letter “A” depicts a double bond, then R20 is absent;
when R26 is not oxo and the dotted line that is labeled with the letter “A” depicts a single bond, then R20 is selected from hydro; 4-hydroxyphenyl; (2-hydroxyphenyl)methyl; and (3,4-dihydroxyphenyl)methyl;
when R27 is oxo, then R29 is absent; and
when R27 is not oxo, then R29 is selected from hydro; methyl; 4-methylpent-3-enyl; 4,8,12-trimethyltridecyl; and 4,8,12-trimethyltrideca-3,7,11-trieneyl.

94. The method of any one of claims 80-86 and 93, wherein the molecule is alkannin; atovaquone; buparvaquone; cannabichromene; dehydroequol; glabrene; gossypol; javanicin; juglone; lapachol; lawsone; menadiol; naphthazarin; naphthoresorcinol; 2-naphthol; phthiocol; phylloquinol; plumbagin; PSB-SB-487; sappanol; spinochrome B; or uliginosin B.

95. The method of any one of claims 80-86 and 93, wherein the molecule is alpha-tocopherol; beta-tocopherol; gamma-tocopherol; delta-tocopherol; zeta2-tocopherol; eta-tocopherol; alpha-tocotrienol; beta-tocotrienol; gamma-tocotrienol; or delta-tocotrienol.

96. The method of any one of claims 80-86, wherein the anion has a general structure IIIa or IIIb 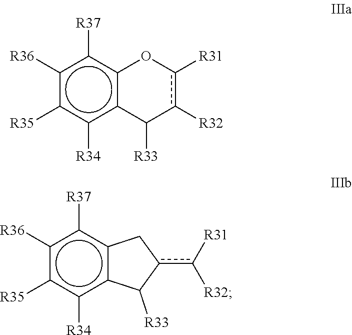
one of R31 and R32 is selected from phenyl; 2-hydroxyphenyl; 3-hydroxyphenyl; 4-hydroxyphenyl; 2,4-dihydroxyphenyl; 2,6-dihydroxyphenyl; 3,4-dihydroxyphenyl; 3,5-dihydroxyphenyl; 3,4,5-trihydroxyphenyl; 3-hydroxy-4-methoxyphenyl; 4-hydroxy-3-methoxyphenyl; 3,4-dihydroxy-5-methyoxyphenyl; 3,5-dihydroxy-4-methyoxyphenyl; 3-hydroxy-4,5-dimethoxyphenyl; 4-hydroxy-3,5-dimethoxyphenyl; 2-methoxyphenyl; 3-methoxyphenyl; 4-methoxyphenyl; 2,4-dimethoxyphenyl; 2,6-dimethoxyphenyl; 3,4-dimethoxyphenyl; 3,5-dimethoxyphenyl; 3,4,5-trimethoxyphenyl; (3,4-dihydroxyphenyl)methyl; 1,3-benzodioxol-5-yl; 4,6-dimethoxy-3,5,11-trimethyltrideca-7,9,11-trienyl; 5-hydroxy-3,4-dimethoxybicyclo[4.2.0]octa-1,3,5-triene-1,7-diyl; and 2-oxo-3-hydroxy-10-(4-hydroxy-3-methoxyphenyl)-4-oxatricyclo[4.3.1.0^{3,7}]dec-8-en-8-yl, and the other one of R31 and R32 is selected from hydro; methyl; isoprenyl; geranyl; hydroxy; methoxy; and [(3,4,5-trihydroxyphenyl)carbonyl]oxy;
R33 is selected from hydro; hydroxy; and oxo;
one of R34, R35, R36, and R37 is an oxide group, and the other three of R34, R35, R36, and R37 are each independently selected from hydro; methyl; isoprenyl; geranyl; 5-methyl-2-(prop-1-en-2-yl)hex-4-enyl; 3,7-dimethylocta-2,6-dien-1-yl; hydroxy; and methoxy; and
each dotted line depicts an optional double bond.

97. The method of any one of claims 80-86 and 96, wherein the molecule is acacetin; acerosin; afzelechin; alnetin; ampelopsin; apiforol; apigenin; aromadendrin; artocarpetin; aureusidin; axillarin; azaleatin; baicalein; baptigenin; biochanin A; blumeatin; butin; calycosin; cannflavin A; cannflavin B; cannflavin C; catechin; chrysin; chrysoeriol; cirsilineol; cirsiliol; cirsimaritin; corymbosin; coumafuryl; daidzein; datiscetin; derrubone; dihydrokaempferide; dihydrokaempferol; dihydromorin; 4′,7-dihydroxyflavone; 7,8-dihydroxyflavone; diosmetin; echioidinin; epiafzelechin; epicatechin; epicatechin gallate; epigallocatechin; epigallocatechin gallate; equol; eriodictyol; ermanin; eupatilin; eupatorin; FBL-03G; fisetin; fisetinidol; formononetin; fustin; galangin; gallocatechin; garbanzol; gardenin A; gardenin B; gardenin C; gardenin D; gardenin E; genistein; 5-O-methylgenistein; genkwanin; geraldone; glycitein; gossypetin; guibourtinidol; hesperetin; hispidulin; homoeriodictyol; 6-hydroxyflavone; hymenoxin; hypolaetin; irigenin; isorhamnetin; isosakuranetin; isoscutellarein; isosilychristin; isoxanthohumol; jaceosidin; kaempferol; 4′ methylkaempferol; laricitrin; leucocyanidin; leucofisetinidin; leucopelargonidin; leucopeonidin; liquiritigenin; luteoforol; luteolin; 6-hydroxyluteolin; luteone; mearnsetin; meciadanol; melacacidin; mesquitol; methylchrysin; mikanin; morin; myricetin; naringenin; negletein; nepetin; nevadensin; nodifloretin; norartocarpetin; norwogonin; 0-806; onopordin; oritin; orobol; oroxylin A; ourateacatechin; pachypodol; pectolinarigenin; pedalitin; pilloin; pinobanksin; pinocembrin; pinostrobin; poriol; pratensein; pratol; primetin; primuletin; prunetin; pseudobaptigenin; psi-tectorigenin; quercetagetin; quercetin; retusin; rhamnazin; rhamnetin; robinetinidol; sakuranetin; scaposin; scillavone A; scillavone B; scutellarein; serpyllin; silychristin; silydianin; sophoraflavanone G; sorbifolin; spinacetin; sterubin; stigmatellin; sudachitin; sulfuretin; syringetin; taxifolin; techtochrysin; tectorigenin; tithonine; tricetin; tricin; velutin; wighteone; wightin; wogonin; xanthomicrol; or zapotinin.

98. The method of any one of claims 80-86, wherein the anion has a general structure IV 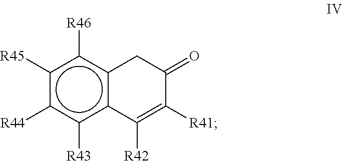
exactly one of R41, R42, R43, R44, R45, and R46 is an oxide group;
exactly one of R41, R42, R43, R44, R45, and R46 is selected from hydro; isoprenyl; geranyl; 1-phenylpropyl; (3,7-dimethyloct-2-enyl)oxy; 3-oxo-1-(furan-2-yl)butyl; (2-oxo-4-hydroxy-2H-chromen-3-yl)methyl; 1-(2-oxo-4-hydroxy-2H-chromen-3-yl)ethyl; 2-oxo-2-ethoxy-1-(2-oxo-4-hydroxy-2H-chromen-3-yl)ethyl; 3-hydroxy-3-[4-(4-bromophenyl)phenyl]-1-phenylpropyl; 3-hydroxy-3-(4-chlorophenyl)-1-(5-chlorothiophene-2-yl)propyl; 1,2,3,4-tetrahydronaphthalen-1-yl; 3-(4-phenyl)phenyl-1,2,3,4-tetrahydronaphthalen-1-yl; 3-[4-(4-bromophenyl)phenyl]-1,2,3,4-tetrahydronaphthalen-1-yl; and 3-[4-({[4-(trifluoromethyl)phenyl]methyl}oxy)phenyl]-1,2,3,4-tetrahydronaphthalen-1-yl; and
every other one of R41, R42, R43, R44, R45, and R46 is selected from hydro; hydroxy; methyl; and methoxy.

99. The method of any one of claims 80-86 and 98, wherein the molecule is brodifacoum; bromadiolone; coumatetralyl; daphnetin; dicoumarol; difenacoum; esculetin; ethyl biscoumacetate; ethylidene dicoumarol; ferujol; flocoumafen; fraxetin; 4-hydroxycoumarin; hymecromone; ostruthin; phenprocoumon; scopoletin; tioclomarol; or umbelliferone.

100. The method of any one of claims 80-86, wherein the anion has a general structure Va or Vb 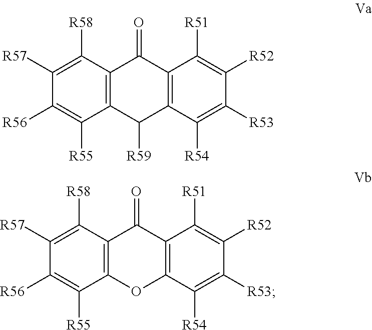
exactly one of R51, R52, R53, R54, R55, R56, R57, and R58 is an oxide group; exactly one or two of R51, R52, R53, R54, R55, R56, R57, and R58 is selected from hydro, isoprenyl, and geranyl; and every other one of R51, R52, R53, R54, R55, R56, R57, and R58 is selected from hydro, hydroxy, methyl, hydroxymethyl, methoxy, and formyl; and
R59 is selected from hydro and oxo.

101. The method of any one of claims 80-86 and 100, wherein the molecule is alizarin; alizarin 1-methyl ether; alizarin 2-methyl ether; aloe emodin; anthragallol; anthralin; anthrapurpurin; 1,6-dihydroxyanthraquinone; anthrarobin; anthrarufin; beta-mangostin; chrysarobin; 9-hydroxychrysarobin; 3-hydroxychrysazin; damnacanthal; danthron; emodin; euxanthone; flavopurpurin; gamma-mangostin; 3,6-dimethylmangostin; 6-deoxy-gamma mangostin; gentisin; mangostin; oxyanthrarufin; oxychrysazin; parietin; purpurin; purpurin 1-methyl ether; purpurin 2,4-dimethyl ether; purpurin 2-methyl ether; purpuroxanthin; quinalizarin; quinizarin; or rubiadin.

102. The method of any one of claims claim 80-86, wherein the anion has a general structure VI 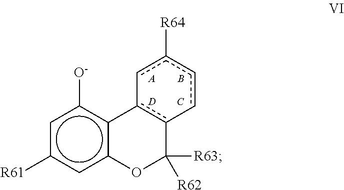
R61 is selected from hydro; methyl; ethyl; propyl; butyl; pentyl; hexyl; heptyl; octyl; nonyl; decyl; prop-2-yl; but-2-yl; pent-2-yl; hex-2-yl; hept-2-yl; octan-2-yl; nonan-2-yl; decan-2-yl; 2-methylpropyl; 2-methylbutyl; 2-methylpentyl; 2-methylhexyl; 2-methylheptyl; 2-methyloctyl; 2-methylnonyl; 2-methyldecyl; 2-methylprop-2-yl; 2-methylbut-2-yl; 2-methylpent-2-yl; 2-methylhex-2-yl; 2-methylhept-2-yl; 2-methyloctan-2-yl; 2-methylnonan-2-yl; 2-methyldecan-2-yl; 3-methylbut-2-yl; 3-methylpent-2-yl; 3-methylhex-2-yl; 3-methylhept-2-yl; 3-methyloctan-2-yl; 3-methylnonan-2-yl; 3-methyldecan-2-yl; 2,3-dimethylbut-2-yl; 2,3-dimethylpent-2-yl; 2,3-dimethylhex-2-yl; 2,3-dimethylhept-2-yl; 2,3-dimethyloctan-2-yl; 2,3-dimethylnonan-2-yl; 2,3-dimethyldecan-2-yl; cyclopropyl; 1-methylcyclopropyl; 1-ethylcyclopropyl; 1-propylcyclopropyl; 1-butylcyclopropyl; 1-pentylcyclopropyl; 1-hexylcyclopropyl; 1-heptylcyclopropyl; 1-octylcyclopropyl; 1-nonylcyclopropyl; cyclobutyl; 1-methylcyclobutyl; 1-ethylcyclobutyl; 1-propylcyclobutyl; 1-butylcyclobutyl; 1-pentylcyclobutyl; 1-hexylcyclobutyl; 1-heptylcyclobutyl; 1-octylcyclobutyl; cyclopentyl; 1-methylcyclopentyl; 1-ethylcyclopentyl; 1-propylcyclopentyl; 1-butylcyclopentyl; 1-pentylcyclopentyl; 1-hexylcyclopentyl; 1-heptylcyclopentyl; cyclohexyl; 1-methylcyclohexyl; 1-ethylcyclohexyl; 1-propylcyclohexyl; 1-butylcyclohexyl; 1-pentylcyclohexyl; 1-hexylcyclohexyl; ethenyl; prop-1-enyl; but-1-enyl; pent-1-enyl; hex-1-enyl; hept-1-enyl; octan-1-enyl; nonan-1-enyl; decan-1-enyl; ethynyl; prop-1-ynyl; but-1-ynyl; pent-1-ynyl; hex-1-ynyl; hept-1-ynyl; octan-1-ynyl; nonan-1-ynyl; decan-1-ynyl; 2-phenylethyl; 2-phenylprop-2-yl; adamant-1-yl; adamant-2-yl; 6-bromohex-2-enyl; 6-bromohex-2-ynyl; 2-methyl-6-bromohex-2-yl; 6-cyanohex-2-enyl; and 6-cyanohex-2-ynyl;
R62 is selected from hydro and methyl;
R63 is selected from hydro; methyl; 3-hydroxypropyl; 3-hydroxyprop-1-enyl; and 3-hydroxyprop-1-ynyl;
R64 is selected from hydro; methyl; hydroxy; hydroxymethyl; and oxo; and
the dotted lines that are labeled with A, B, C, and D depict four optional double bonds that are selected such that A, B, C, and D depict either (i) zero double bonds; (ii) one double bond that occurs at the dotted line that is labeled with either A, B, or D; or (iii) three double bonds that occur at the dotted lines that are labeled with A, C, and D.

103. The method of any one of claims 80-86 and 102, wherein the molecule is AM-087; AM-2389; AM-4030; AM-411; AM-905; AM-906; AM-919; AM-938; AMG-1; AMG-36; AMG-41; canbisol; cannabinol; 11-nor-9beta-hydroxyhexahydrocannabinol; delta8-tetrahydrocannabinol; dexanabinol; dimethylheptylpyran; HU-210; HU-243; KM-233; nabilone; perrottetinene; synhexyl; tetrahydrocannabinol; 11-hydroxytetrahydrocannabinol; tetrahydrocannabinol-C4; tetrahydrocannabiorcol; tetrahydrocannabiphorol; or tetrahydrocannabivarin.

104. The method of any one of claims 80-86, wherein the anion has a general structure VIIa or VIIb 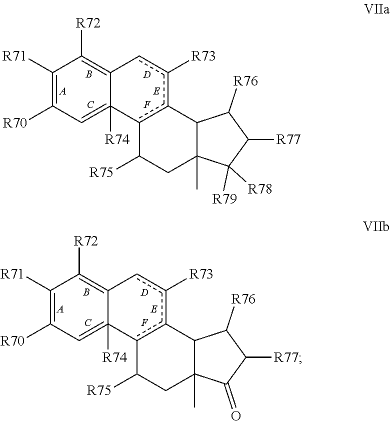
R70 is selected from hydro, formyl, and cyano;
R71 is selected from oxo and oxide;
when R70 is formyl or cyano, then R71 is oxide, R72 is hydro, and the dotted lines that are labelled with A, B, C, D, E, and F depict a single double bond that occurs at A;
when R70 is hydro and R71 is oxo, then R72 is oxide, and the dotted lines that are labelled with A, B, C, D, E, and F depict a single double bond that occurs at B;
when R70 is hydro and R71 is oxide, then R72 is selected from is hydro and hydroxy, and the dotted lines that are labelled with A, B, C, D, E, and F depict either (i) three double bonds that occur at A, B, and C, (ii) four double bonds that occur at either A, B, C, and E or A, B, C, and F, or (iii) five double bonds that occur at A, B, C, D, and F;
R73 is selected from hydro and 9-[(4,4,5,5,5-pentafluoropentyl)sulfinyl]nonanyl;
R74 is selected from hydro and methyl;
R75 is selected from hydro, hydroxy, and methoxy;
R76 and R77 are each independently selected from hydro and hydroxy;
R78 is selected from hydro, methyl, ethyl, ethenyl, and ethynyl; and
R79 is selected from hydroxy, acetyloxy; (1-oxopropyl)oxy; (1-oxobutyl)oxy; (1-oxopentyl)oxy, (1-oxohexyl)oxy; (1-oxoheptyl)oxy, (1-oxooctanyl)oxy, (1-oxononanyl)oxy, (1-oxodecanyl)oxy, (1-oxoundecanyl)oxy, [1-oxo-3-(cyclopentyl)propyl]oxy, and [(2-methylprop-2-yl)amino]carbonyl.

105. The method of claim 104, wherein R71 is oxide; R70 and R72 are hydro; and the dotted lines labeled with A, B, and C depict exactly 3 double bonds.

106. The method of any one of claim 80-86 or 104, wherein the molecule is 17alpha-dihydroequilin; 17beta-dihydroequilin; 16-epiestriol; 17-epiestriol; 16,17-epiestriol; equilenin; equilin; estradiol; alpha-estradiol; estriol; estrone; ethinyl estradiol; formestane; fulvestrant; isoestradiol; 8-isoestrone; moxestrol; oxabolone; oxymesterone; or oxymetholone.

107. The method of any one of claim 80-86 or 104, wherein the molecule is estradiol enanthate; estradiol undecylate; estradiol valerate; estradiol 17beta-cyclopentanepropanoate; or oxabolone 17-cyclopentanepropionate.

108. The method of any one of claims 80-86, wherein the anion has a general structure VIIIa, VIIIb, VIIIc, VIIId, VIIIe, or VIIIf 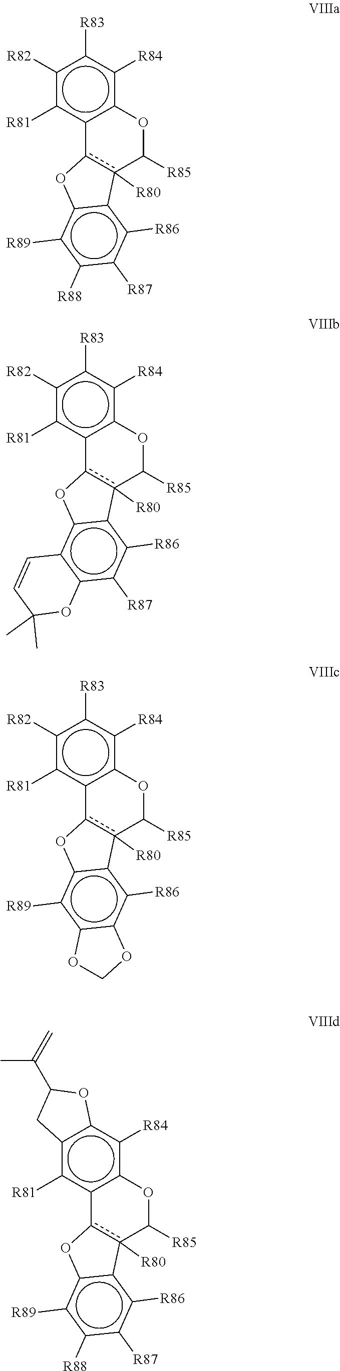 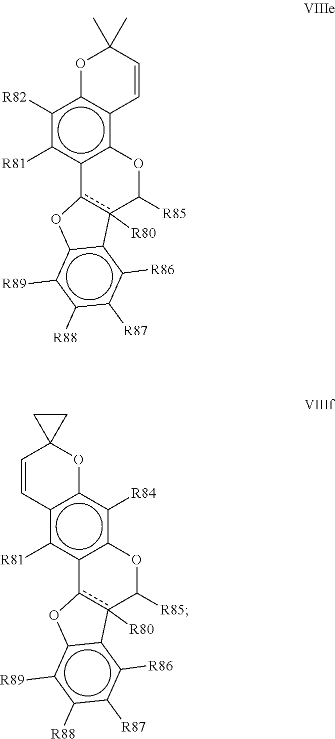
each dotted line depicts an optional double bond, wherein when the double bond is selected, then R80 is absent; and when the double bond is not selected, then R80 is selected from hydro and hydroxy;
general structures VIIIa, VIIIb, VIIIc, VIIId, VIIIe, and VIIIf each comprise one oxide group, wherein one of R81, R82, R83, R84, R86, R87, R88, and R89 is oxide; one or two of R81, R82, R83, R84, R86, R87, R88, and R89 are selected from hydro, isoprenyl, geranyl, and 4-hydroxy-3-methylbutyl; and every other one of R81, R82, R83, R84, R86, R87, R88, and R89 is selected from hydro, hydroxy, and methoxy; and
when R80 is absent or hydro, then R85 is selected from hydro, hydroxy, and oxo; and when R80 is hydroxy, then R85 is hydro.

109. The method of claim 108, wherein:
when the double bond is selected, then R85 is selected from hydro and oxo; and when the double bond is not selected, then R85 is hydro;
either R83 is oxide and R88 is hydroxy or methoxy; R88 is oxide and R83 is hydroxy or methoxy; or R83 and R88 are each independently selected from hydroxy and methoxy; and
R86 is hydro.

110. The method of any one of claims 80-86, 108, and 109, wherein the molecule is cabenegrin A-I, cabenegrin A-II, coumestrol, glyceollin I, glyceollin II, glyceollin III, glyceollin IV, glycinol, glycyrrhizol A, medicagol, medicarpin, phaseolin, plicadin, psoralidin, or wedelolactone.

111. The method of any one of claims 80-86, wherein the anion has a general structure IXa, IXb, IXc, IXd, IXe, IXf, or IXg 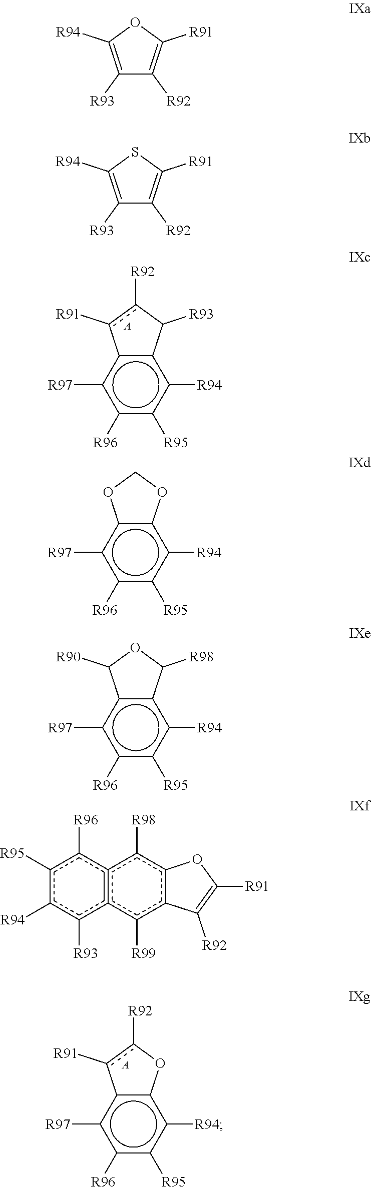
general structures IXa, IXb, IXc, IXd, IXe, IXf, and IXg each comprise an oxide group, wherein one of R91, R92, R93, R94, R95, R96, and R97 is oxide; two of R91, R92, R93, R94, R95, R96, and R97 are independently selected from hydro, ethyl, propyl, prop-2-yl, ethenyl, prop-1-enyl, prop-2-enyl, propen-2-yl, 2-methylprop-1-enyl, isoprenyl, formyl, acetyl, (ethoxy)carbonyl, (propoxy)carbonyl, 2-hydroxprop-2-yl, phenyl, 4-hydroxyphenyl, 3,4-dihydroxyphenyl, 3-hydroxy-4-methoxyphenyl, 4-hydroxy-3-methoxyphenyl, 4-oxo-3,5,7-trihydroxy-2,3-dihydro-4H-chromen-2-yl, 2-[1-oxo-7,8-dihydroxy-3-(methoxycarbonyl)-1H-isochromen-6-yl]ethyl, fluoro, chloro, bromo, and iodo; and every other one of R91, R92, R93, R94, R95, R96, and R97 is selected from hydro, hydroxy, hydroxymethyl, methoxy, and methyl;
R90 is selected from hydro, hydroxy, oxo, (phenyl)methylidene, (4-hydroxyphenyl)methylidene, and (3,4-hydroxyphenyl)methylidene;
R98 is selected from hydro, hydroxy, and oxo; when R98 is oxo, then R99 is oxo; and when R98 is hydro or hydroxy, then R99 is independently selected from hydro and hydroxy;
each dotted line labeled with “A” is an optional double bond;
general structure IXf comprises exactly 12 implicit carbon atoms that are each depicted by a junction of three lines in general structure IXf; and
each dotted line in general structure IXf depicts an optional double bond that is selected such that each of the 12 implicit carbon atoms is bonded to exactly one other atom of general structure IXf with a double bond.

112. The method of any one of claim 80-86 or 111, wherein the molecule is chlorindanol; collinomycin; euparin; isomaltol; isosilychristin; mutisianthol; protiofate; sesamol; silychristin; or thunberginol F.

113. The method of any one of claims 1-112, wherein the composition comprises water and hydroxide.

114. The method of any one of claims 1-113, wherein the composition comprises ethanol and ethoxide.

115. The method of any one of claims 1-114, wherein the composition comprises 1,2-propanediol and one or both of 1-hydroxypropane-2-oxide and 2-hydroxypropane-1-oxide.

116. The method of any one of claims 1-114, wherein the composition comprises 1,2,3-propanetriol and one or both of 1,3-dihydroxypropane-2-oxide and 2,3-dihydroxypropane-1-oxide.

117. The method of any one of claims 1-116, wherein the composition comprises lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); copper (II) cation (“Cu++”); ammonium (“NH₄+”); protonated ethanolamine; choline; protonated lysine; protonated arginine; or protonated sphingosine.

118. The method of any one of claims 1-117, wherein the composition comprises sodium cation.

119. The method of any one of claims 1-118, wherein the composition comprises potassium cation.

120. The method of any one of claims 1-119, wherein the subject is human.
